(19) United States
(12) Patent Application Publication
(10) Pub. No.: US 2007/0232589 A1
(43) Pub. Date: Oct. 4, 2007

(54) Pharmaceutical Composition And Method For Treating Neurodegenerative Disorders

(75) Inventor: Adrian Hobden, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/674,032

(22) Filed: Feb. 12, 2007

Related U.S. Application Data
(63) Continuation of application No. PCT/US05/28716, filed on Aug. 11, 2005.
(60) Provisional application No. 60/600,600, filed on Aug. 11, 2004.

(51) Int. Cl. A61K 31/55; C07D 223/14

(52) U.S. Cl. 514/215; 540/576

(57) ABSTRACT

The invention provides compositions and methods for treating neurodegenerative disorders. The method of the invention involves administering to an individual in need of treatment a composition an acetylcholine esterase inhibitor in combination with another therapeutic agent. The methods and compositions of the invention are useful for treating and preventing neurodegenerative disorders like Alzheimer's disease, dementia, and mild cognitive impairment.

### CROSS-REFERENCE TO RELATED APPLICATIONS

This application claims priority international application no. PCT/US2005/028716, filed Aug. 11, 2005 (publication no. WO2006/020852), which claims priority to U.S. Provisional Application Ser. No. 60/600,600 filed on Aug. 11, 2004, both of which are incorporated herein by reference in their entireties.

### TECHNICAL FIELD OF THE INVENTION

The invention provides a method for the therapeutic treatment of neurodegenerative disorders. The invention further provides a method for prophylaxis against neurodegenerative disorders. The invention further provides pharmaceutical composition for use in the methods of the invention. The invention has utility for treating and preventing neurodegenerative disorders such as Alzheimer's disease, dementia, and mild cognitive impairment.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors. In the United States alone, four million adults suffer from Alzheimer's disease (AD). Not only does Alzheimer's disease significantly impact the lives of countless families today, it threatens to become even more of a problem as the baby boom generation matures. The economic burden of AD in the United States is estimated to cost over $100 billion a year and the average lifetime cost per patient is estimated to be $174,000. Unfortunately, there is no cure available for AD.

In 1906, Dr. Alois Alzheimer, noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause in decline of cognitive function. The number of tangles is more highly correlated with the cognitive decline than amyloid load in patients with AD (Albert Proc. Natl. Acad. Sci. U.S.A. 93:13547-13551 (1996)). The cellular, biochemical, and molecular events responsible for neuronal and synaptic loss in AD are not known. A number of studies have demonstrated that amyloid can be directly toxic to neurons (Iversen et al. Biochem. J. 311:1-16 (1995); Weiss et al. J. Neurochem. 62:372-375 (1994); Lorenzo et al. Ann. N.Y. Acad. Sci. 777:89-95 (1996); Storey et al. Neuropathol. Appl. Neurobiol. 2:81-97 (1999)), resulting in behavioral impairment. The toxicity of amyloid or tangles is potentially aggravated by activation of the complement cascade (Rogers et al. Proc. Natl. Acad. Sci. U.S.A. 21:10016-10020 (1992); Rozemuller et al. Res. Immunol. 6:646-9 (1992); Rogers et al. Res. Immunol. 6:624-30 (1992); Webster et al. J. Neurochem. 69(1):388-98 (1997)). This suggests involvement of an inflammatory process in AD and neuronal death seen in AD (Fagarasan et al Brain Res. 723(1-2):231-4. (1996); Kalaria et al. Neurodegeneration 5(4):497-503 (1996); Kalaria et al. Neurobiol Aging. 17(5):687-93 (1996); and Farlow Am. J. Health Syst. Pharm. 55 Suppl. 2:S5-10 (1998)).

Evidence that amyloid β protein (Aβ) deposition causes some forms of AD was provided by genetic and molecular studies of some familial forms of AD (FAD). (See, e.g., Ii Drugs Aging 7(2):97-109 (1995); Hardy Proc. Natl. Acad. Sci. U.S.A. 94(6):2095-7 (1997); Selkoe J. Biol. Chem. 271(31):18295-8 (1996)). The amyloid plaque buildup in AD patients suggests that abnormal processing of Aβ may be a cause of AD. Aβ is a peptide of 39 to 42 amino acids and forms the core of senile plaques observed in all Alzheimer cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in these FAD genes can result in increased Aβ deposition.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ₄₂, both in model transfected cells and transgenic animals. The other two FAD genes code for presenilin 1 and 2 (PS1, PS2) (Hardy Proc. Natl. Acad. Sci. U.S.A. 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP gene, and all of them also cause FAD with 100% penetrance. Similar to APP mutants, studies have demonstrated that PS1 and PS2 mutations shift APP metabolism, resulting in elevated Aβ₄₂ production (in vitro and in vivo).

Cyclooxygenases (COX) are major Alzheimer's disease drug targets due to the epidemiological association of NSAID use, whose primary target are cycloxygenases, with a reduced risk of developing Alzheimer's disease (see, e.g., Hoozemans et al. Curr. Drug Targets 4(6):461-8 (2003) and Pasinetti et al. J. Neurosci. Res. 54(1):1-6 (1998)). The epidemiological studies have indicated that chronic NSAID use appears to reduce the risk of acquiring Alzheimer's disease and/or delay the onset of the disease (see e.g., McGeer et al. Neurology 47(2):425-432 (1996); and Etminan et al. BMJ. 327(7407):128 (2003)). COX-2 selective inhibitors are attractive candidates for long-term drug use since they do not inhibit COX-1 and appear to be less toxic. In support of COX-2 as a target for the treatment for AD, a recent study was published reporting that in mouse models of AD, COX-2 overexpression was related to the neuropathology of AD (Xiang et al. Neurobiol. Aging 23:327-34 (2002)). However, recent clinical trials of specific NSAIDs have called into question the hypothesis the hypothesis that anti-inflammatory drugs are useful for the treatment or prevention of Alzheimer's disease. It was reported that rofecoxib, a COX-2 selective NSAID, at 25 mg daily, failed to show efficacy for treating AD. Naproxen, another NSAID, in the same trial failed to show efficacy in Alzheimer's treatment. See Aisen et al. JAMA 289:2819-26 (2003) and Reines et al. Neurology 62(1):66-71 (2004). These authors concluded that the results with naproxen and rofecoxib do not support the use of NSAIDs for the treatment of AD. Celecoxib, a COX-2-selective NSAID, failed to show efficacy in several recent clinical trials for the treatment of AD. See Jhee et al., “A Double-Blind, Placebo-Controlled Pharmacokinetic (PK), Pharmacodynamic (PD) and Safety Study of Celecoxib Treatment for Four Weeks in Patients with Alzheimer's Disease (AD),” Abstract from 7^{th} International Geneva/Springfield Symposium on Advances in Alzheimer's Therapy (2002); also published in Clinical Research and Regulatory Affairs 21(1): 49-66 (2004) and Sainati et al. (Abstract from 6^{th} International Stockholm/Springfield Symposium on Advances on Alzheimer's Therapy, Abstract Book 2000; 180). Conversely, it was reported recently that rofecoxib provides neuroprotection in an in vivo Alzheimer's disease excitotoxic model system (Scali et al. Neuroscience 117:909-919 (2003)). However, rofecoxib, in a large prevention clinical trial, failed to prevent the development of Alzheimer's disease in patients having mild cognitive impairment. In fact, the results of this trial showed that 6.4% of patients taking rofecoxib developed AD as compared to 4.5% for those taking placebo (see e.g., Visser et al., abstract from Annual meeting of the American College of Neuropsychopharmacology San Juan, Puerto Rico, 2003; and Landers, Wall Street Journal 10 Dec. 2003). Thus, clinical trials have indicated that NSAIDs, as a general class of drugs, are not likely to be useful for treating and/or preventing Alzheimer's disease.

Aβ formation is another target for affecting Alzheimer's disease progression since Aβ amyloid plaques are a central pathological hallmark of the disease. Recently, it was suggested that certain NSAIDs are capable of lowering the level of Aβ₄₂, the form of Aβ associated with plaque formation. U.S. Patent Application 2002/0128319 to Koo et al., U.S. Application Publication No. 2002/0128319, discloses the use of an Aβ₄₂ lowering amount of NSAID for treating Alzheimer's disease. (R)-2-(2-fluoro-4-biphenylyl)propionic acid, which negligibly inhibits COX activity, was reported in Koo et al. to lower Aβ₄₂ in a transgenic mouse model and CHO cells.

A recent clinical trial using a therapy designed to eliminate Aβ plaques from disease patients failed despite strong evidence of efficacy in animal models (Pfiefer et al. Science 298:1379 (2002)). The Aβ-lowering therapy that worked in animal models caused serious problems in humans. In view of the clinical studies, Atwood et al. (Science 299:1014 (2003)) noted that “[m]ounting evidence indicates that this deposition of amyloid-β may be a neuroprotective response to injury” and “[t]hese results demonstrate yet again the futility of removing a protein, amyloid-β, which has ubiquitous tissue expression, without first understanding its function(s).”

Additionally, gamma-secretase inhibitors, which were designed to alter processing of APP, have turned out to be toxic compounds not likely to be suitable for chronic human use. See De Strooper et al. Nature 398:518-522 (1999); Wong et al. J. Biol. Chem. 279:12876-12882 (2004); and Hadland et al. PNAS 98(13):7487-91 (2001). Thus, it is not clear if gamma-secretase inhibitors are a realistic treatment/prevention option. Indeed, as noted recently, mutations in PS-1 associated with AD may cause the disease not through altering Aβ processing, but rather by affecting calcium homeostasis (Mattson, Nature 442:385-386 (2003)).

Several epidemiological studies have reported an association between long-term use of NSAIDs, such as ibuprofen and aspirin, with reduced risk for certain malignancies and neurodegenerative processes characterized by dementia of the Alzheimer's type. A variety of explanations have been given for the reduced cancer and Alzheimer's disease (AD) risk associated with long-term NSAID use. The primary action of NSAIDs appears to be inhibition of cyclooxygenase (COX) activity. Thus, a leading hypothesis is that NSAIDs reduce risk for certain cancers and Alzheimer's disease by affecting the COX enzymes. Other explanations include mediation of apoptosis, modulation of growth factors, and modulation of the nuclear factor kappa B pathway (NF-κB).

U.S. Pat. No. 5,192,753 to Rogers et al alleges NSAIDs are useful for treating Alzheimer's disease through the inhibition of cyclooxygenase and therefore inhibition of prostaglandin synthesis. U.S. Pat. No. 5,643,960 to Brietner et al. reports the use of COX inhibiting NSAIDs to delay the onset of Alzheimer's symptoms. U.S. Pat. No. 6,025,395 to Brietner et al. relates to the use of COX inhibiting NSAIDs.

Flurbiprofen is a racemic non-steroidal anti-inflammatory drug (NSAID) having a chemical name of (R,S)-2-(2-fluoro-4-biphenylyl)propionic acid. 50 milligram (mg) and 100 mg racemic flurbiprofen tablets are marketed as ANSAID® and FROBEN® for the treatment of chronic inflammatory disease.

The literature has described a variety of (R)-2-(2-fluoro-4-biphenylyl)propionic acid-containing compositions. Brune et al. J. Clin. Pharmacol. 32:944-952 (1992) discloses the use of tablets containing 50 mg of (R)-2-(2-fluoro-4-biphenylyl)propionic acid. Jerussi et al. (J. Clin. Pharmacol. 32:944-952 (1992)) describe the use of 100 mg b.i.d. (R)-2-(2-fluoro-4-biphenylyl)propionic acid in investigating gastroduodenal tolerance. Lotsch et al. (Bri. J. Clin. Pharm. 40:339-346 (1995) describe the use 50 mg and 100 mg doses of (R)-2-(2-fluoro-4-biphenylyl)propionic acid in pain related chemo-somatosensory evoked potentials in human subjects. The authors concluded that (R)-2-(2-fluoro-4-biphenylyl)propionic acid, at these doses, produced an analgesic effect. Geisslinger et al. (Br. J. Clin. Pharmacol. 37(4):392-4 (1994)) discloses the use of 50 mg (R)-2-(2-fluoro-4-biphenylyl)propionic acid for examining the disposition of single enantiomers in humans. Oelkers et al. (Br. J. Clin. Pharmacol. 43(2):145-53 (1997)) disclose the use of 75 mg (R)-2-(2-fluoro-4-biphenylyl)propionic acid for studying its effects and disposition in blister fluid and human serum. U.S. Pat. No. 5,206,029 to Brune et al. discloses medicaments, containing 10 to 100 mg doses of previously separated flurbiprofen enantiomers, in ratios of from 99.5%:0.5% to 0.5%:99.5%, that are effective for treating pain and inflammatory conditions. U.S. Pat. No. 5,200,198 to Geisslinger et. al. discloses a medicament, containing 10 to 100 mg doses of substantially pure (R)-2-(2-fluoro-4-biphenylyl)propionic acid and mixtures containing up to 40% S-enantiomer, that are effective for treating pain and inflammatory conditions.

Of the five drugs currently being used in the US for the treatment of AD, four of them—tacrine (Cognex®), donepezil (Aricept®), rivastigmine (Exelon®), and galantamine (Reminyl® now known as Razadyne®)—are inhibitors of acetylcholine esterase. Another drug, memantine, was recently approved for treating moderate-to-severe AD. More recently it was reported that memantine showed efficacy in treating mild-to-moderate AD. Memantine is a NMDA receptor antagonist.

The drugs currently used for treating AD, including memantine and the acetylcholine esterase inhibitors, are marginally efficacious and have undesirable side-effects. Thus, there is a large unmet need for better and safer drugs.

### SUMMARY OF THE INVENTION

The invention generally relates to compositions and therapeutic treatments for neurodegenerative disorders. More specifically, the invention provides a pharmaceutical composition for treating and/or preventing neurodegenerative disorders. The composition of the invention has (1) an acetylcholine esterase inhibitor, (2) one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid, and Aβ42 lowering agents, and (3) one or more pharmaceutically acceptable carriers (excipients). The method of the invention involves administering, to an individual in need of treatment, a therapeutically effective amount of an acetylcholine esterase inhibitor and one or more compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents.

In a first embodiment, the invention provides a composition comprising a first compound that is acetylcholine esterase inhibitor (or a pharmaceutically acceptable salt, ester, or prodrug thereof) and one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid, and Aβ42 lowering agents (or a pharmaceutically acceptable salt, ester, or prodrug thereof). According to this embodiment, the acetylcholine esterase inhibitor is galantamine. In one aspect of this embodiment the one or more second compounds are chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid (or a pharmaceutically acceptable salt, ester, or prodrug thereof). In another aspect, the second compound is (R)-2-(2-fluoro-4-biphenylyl)propionic acid (or a pharmaceutically acceptable salt, ester, or prodrug thereof). In another aspect, the second compound is chosen from Aβ42 lowering agents. The compositions of this embodiment can provide the two components together in a single unit dosage form with a pharmaceutically acceptable carrier. In some aspects of this embodiment, the unit dosage form is chosen from a tablet, a capsule, or a caplet unit dosage form.

In a second embodiment, the invention provides a method for treating neurodegenerative disorders. According to the method of this embodiment, a therapeutically effective amount of a first compound which is an acetylcholine esterase inhibitor and one or more seconds compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-Dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents (or a pharmaceutically acceptable salt, ester, or prodrug thereof) and the acetylcholine esterase inhibitor galantamine (or a pharmaceutically acceptable salt, ester, or prodrug thereof) is administered to an individual in need of such treatment. The individual in need of treatment can have a neurodegenerative disorder, a predisposition to a neurodegenerative disorder, and/or desire prophylaxis against neurodegenerative disorders. In one aspect of this embodiment, the therapeutically effective amount of the one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents, and the acetylcholine esterase inhibitor is capable of reducing at least one symptom of the neurodegenerative disorder. In another aspect, for individuals desiring prophylaxis against a neurodegenerative disorder, the effective amount of the one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents, and the acetylcholine esterase inhibitor, is capable of preventing an increase (or slowing the rate of increase) in at least one symptom of the neurodegenerative disorder. According to this embodiment, the acetylcholine esterase inhibitor is galantamine. In one aspect of this method, the one or more second compounds are chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid. In still another aspect of this method, the one or more second compounds is (R)-2-(2-fluoro-4-biphenylyl)propionic acid. In another aspect of this method, the neurodegenerative disease is chosen from Alzheimer's disease, prodromal Alzheimer's disease, mild-to-moderate Alzheimer's disease, moderate-to-severe Alzheimer's disease, dementia, mild Alzheimer's disease, and mild cognitive impairment. In another aspect, the invention provides a method for the treatment or prophylaxis of Alzheimer's disease through the administration of an effective amount of (R)-2-(2-fluoro-4-biphenylyl)propionic acid and galantamine. In some aspects of this embodiment, the lessening in decline in cognitive function is at least 25% as compared to individuals treated with placebo, at least 40%, or at least 60%. For example, an individual treated with placebo having probable mild-to-moderate Alzheimer's disease is expected to score approximately 5.5 points higher on the ADAS-cog test after a specified period of time of treatment (e.g., 1 year) whereas an individual treated with the composition of this aspect of the invention for the same period of time will score approximately 2.2 points higher on the ADAS-cog scale with a 60% decrease in decline or 3.3 points higher with a 40% decrease in decline in cognitive function when treated with the combination of galantamine and the one or more second compounds for the same specified period of time.

In a third embodiment, the invention provides a method of reducing amyloid β₄₂ (Aβ₄₂) protein levels. In particular, the method relates to reducing, lowering, preventing an increase, or slowing the rate of increase in Aβ₄₂ protein levels, in an individual in need of such treatment, by administering to the individual a therapeutically effective amount of the acetylcholine esterase inhibitor galantamine (or a pharmaceutically acceptable salt, ester, or prodrug thereof) and one or more compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents (or a pharmaceutically acceptable salt, ester, or prodrug thereof). The individual in need of treatment can have a neurodegenerative disorder, a predisposition to a neurodegenerative disorder, and/or a desire for prophylaxis against neurodegenerative disorders, where the disorder is characterized by increased Aβ₄₂ protein levels. In one aspect, the effective amount is an amount of galantamine and the one or more second compounds sufficient for reducing Aβ₄₂ protein levels. In another aspect, the effective amount is an amount of galantamine and the one or more second compounds sufficient for reducing Aβ₄₂ protein levels and reducing (or slowing the progression) of one or more symptoms of the neurodegenerative disorder. In another aspect, for individuals desiring prophylaxis against a neurodegenerative disorder, the effective amount is an amount of the acetylcholine esterase inhibitor and one or more second compounds, sufficient for preventing an increase in Aβ₄₂ protein levels or an increase in the rate of Aβ₄₂ increase. In one aspect of this method, the one or more second compounds is chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid. In still another aspect of this method, the second compound is (R)-2-(2-fluoro-4-biphenylyl)propionic acid. The method of the invention further provides for the treatment or prophylaxis of neurodegenerative disorders with an Aβ₄₂ protein lowering effective amount of (R)-2-(2-fluoro-4-biphenylyl)propionic acid and galantamine. In one aspect of this method, the neurodegenerative disease is chosen from Alzheimer's disease, dementia, mild Alzheimer's disease, cerebral amyloid angiopathy, and mild cognitive impairment. In another aspect of this embodiment, the invention provides a method for the treatment or prophylaxis of Alzheimer's disease through the administration, to an individual in need of such treatment, of an Aβ₄₂ protein lowering effective amount of (R)-2-(2-fluoro-4-biphenylyl)propionic acid and galantamine.

In fourth embodiment, the invention provides compositions and a method for treating and/or preventing neurodegenerative disorders by administering, to an individual in need of such treatment, an effective amount of (1) the acetylcholine esterase inhibitor galantamine, (2) one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-Chlorophenyl)benzoxazol-5-yl]propionic acid, (R)-1,8-diethyl-1,3,4,9-tetrahydropyrano(3,4-b)indole-1-acetic acid, and Aβ42 lowering agents (or a pharmaceutically acceptable salt, ester, or prodrug thereof), and (3) one or more compounds selected from the group consisting of secretase inhibitors, GABA-A alpha 5 inverse agonists, NMDA antagonists (i.e., memantine) and antioxidants (or a pharmaceutically acceptable salt, ester, or prodrug thereof). The combination can be administered simultaneously or separately.

In a fifth embodiment, the invention provides a method of lowering Aβ₄₂ levels to a greater extent than inhibiting COX-1, COX-2, or a combination thereof. In particular, the method of this embodiment involves administering to a patient, in need of treatment, an effective amount of the acetylcholine esterase inhibitor galantamine (or a derivative, pharmaceutically acceptable salt, esters, or prodrug thereof) and one or more second compounds (or a pharmaceutically acceptable salt, ester, or prodrug thereof). According to this embodiment, the one or more second compounds are chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents. According to this embodiment, the acetylcholine esterase inhibitor is galantamine. The method of this embodiment involves the lowering (or slowing the rate of increase) of Aβ₄₂ levels while not substantial affecting the activity of COX-1, COX-2, or both COX-1 and COX-2. Thus, the amount that is administered is effective for lowering Aβ₄₂ levels and does not substantially inhibit COX-1, COX-2, or both COX-1 and COX-2. For example, the effective amount can be above the ED₅₀ (the dose therapeutically effective in 50% of the population) for Aβ₄₂ lowering (i.e., slowing rate of increase), and below the ED₅₀ for COX inhibition. Another example is a sufficiently small amount of compound so that inhibition of at least one COX activity is negligible and Aβ₄₂ levels are reduced. The method of this embodiment can be used to treat and/or prevent Alzheimer's disease. The method of this embodiment can also be used to treat and/or prevent MCI, dementia, and other neurodegenerative disorders.

In a sixth embodiment, the invention provides a method for treating a neurodegenerative disorder. According to this embodiment, an individual having Alzheimer's disease, mild-to-moderate Alzheimer's disease, MCI, prodromal Alzheimer's disease, mild Alzheimer's disease, or moderate-to-severe Alzheimer's disease is identified and treated with a combination of (R)-2-(2-fluoro-4-biphenylyl)propionic acid and galantamine. According to this embodiment, the individual is treated with (R)-2-(2-fluoro-4-biphenylyl)propionic acid and galantamine concomitantly in a specified dosing regimen. In one aspect, the individual is treated with galantamine by titrating the daily dose to a selected daily dosage and then the individual is treated with (R)-2-(2-fluoro-4-biphenylyl)propionic acid and galantamine for from about 4 weeks to about one year, after which the individual is treated with (R)-2-(2-fluoro-4-biphenylyl)propionic acid and not galantamine. In another aspect, the individual is treated with galantamine and (R)-2-(2-fluoro-4-biphenylyl)propionic acid concomitantly for a selected period of time, usually for about 4 weeks to about 6 months, although longer periods of combination treatment such as a year or more are included in this embodiment. After combination treatment for the selected period of time, the individual is no longer treated with galantamine, but treatment with (R)-2-(2-fluoro-4-biphenylyl)propionic acid is continued. In a related aspect, an individual having a genetic predisposition to a neurodegenerative disorder is identified and treated with (R)-2-(2-fluoro-4-biphenylyl)propionic acid until the early signs of the neurodegenerative disorder appear. When the early signs of the neurodegenerative disorder appear, e.g., the individual progresses to mild Alzheimer's disease, the individual is then started on a treatment regimen including galantamine and (R)-2-(2-fluoro-4-biphenylyl)propionic acid.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

The invention provides compositions and therapeutic treatments for neurodegenerative disorders. Specifically, the invention provides a composition, for treating and preventing neurodegenerative disorders, having (1) an acetylcholine esterase inhibitor (or a pharmaceutically acceptable salt, ester, or prodrug thereof) and one or more second compounds as described below. The invention provides a method that involves treating an individual in need of treatment with an effective amount of an acetylcholine esterase inhibitor and the one or more second compounds. The method of the invention can involve co-administering the acetylcholine esterase inhibitor and the one or more second compounds, or the acetylcholine esterase inhibitor and the one or more second compounds can be administered to the same individual at different times and/or by different routes of administration. For example, the acetylcholine esterase inhibitor can be administered in the morning and the one or more second compounds can be administered in the evening, or the acetylcholine esterase inhibitor and the one or more second compounds can be administered both twice daily (e.g., in the morning and the evening). The skilled artisan readily recognizes that the invention relates to numerous dosing regimes to accomplish the therapeutic effect. Advantageously, the combination the acetylcholine esterase inhibitor and the one or more second compounds can be administered together as described herein. Without wishing to be bound by theory, it is believed that combination therapy/compositions of the invention can have unexpected properties particularly useful for the treatment and prophylaxis of neurodegenerative disease like dementia, mild cognitive impairment, Alzheimer's disease, mild Alzheimer's disease, and mild-to-moderate Alzheimer's disease.

In one embodiment, the composition of the invention has a therapeutically effective (or a prophylactically effective) amount of (1) an acetylcholine esterase inhibitor (or a pharmaceutically acceptable salt, ester, or prodrug thereof), (2) one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents (or a pharmaceutically acceptable salt, ester, or prodrug thereof), and (3) one or more pharmaceutically acceptable carriers (excipients). The acetylcholine esterase inhibitor used in the invention is galantamine. In one aspect of the invention, the one or more second compounds are chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, and (R)-1,8-diethyl-1,3,4,9-tetrahydropyrano(3,4-b)indole-1-acetic acid (or a pharmaceutically acceptable salt or ester thereof). It is contemplated that nitrosylated and nitrosated prodrugs of the one or more second compounds can also be used in the methods and compositions of the invention (see, e.g., U.S. Pat. Nos. 6,593,347; 5,703,073; and PCT application WO 94/12463 which are herein incorporated by reference in their entirety). In a specific aspect of the invention, the pharmaceutical composition is co-formulated with (R)-2-(2-fluoro-4-biphenylyl)propionic acid and galantamine. In one aspect, the co-formulation is a tablet unit dosage form. In another aspect, the co-formulation is a capsule unit dosage form. In another aspect, the co-formulation is a caplet unit dosage form. In another aspect of this embodiment, the pharmaceutically acceptable excipient is microcrystalline cellulose.

In one embodiment, the composition of the invention has a therapeutically effective (or a prophylactically effective) amount of (1) an acetylcholine esterase inhibitor (or a pharmaceutically acceptable salt, ester, or prodrug thereof), (2) one or more Aβ42 lowering agent (or a pharmaceutically acceptable salt, ester, or prodrug thereof), and one or more pharmaceutically acceptable excipients. In one aspect of this embodiment, the co-formulation is a tablet unit dosage form. In another aspect of this embodiment, the co-formulation is a capsule unit dosage form. In another aspect, the co-formulation is a caplet unit dosage form. In another aspect of this embodiment, the pharmaceutically acceptable excipient is microcrystalline cellulose.

According to one embodiment, the invention provides methods for lowering, preventing an increase, or slowing the rate of increase of Aβ₄₂ levels in an individual in need of such treatment. Thus, by lowering the amounts of Aβ₄₂ (i.e., slowing the rate of increase) in an individual by administering an Aβ₄₂ lowering effective amount of the acetylcholine esterase inhibitor galantamine (or a pharmaceutically acceptable salt, ester, or prodrug thereof) and one or more second compounds chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, (R)-2-[2-(4-chlorophenyl)benzoxazol-5-yl]propionic acid, (R)-1,8-diethyl-1,3,4,9-tetrahydropyrano(3,4-b)indole-1-acetic acid, and Aβ42 lowering agents (or a pharmaceutically acceptable salt, ester, or prodrug thereof), as described herein, that Alzheimer's disease, dementia, and mild cognitive impairment can be treated or prevented. Thus, diseases characterized by increased levels of Aβ₄₂, can be treated or prevented with the methods of this embodiment which are designed to lower Aβ₄₂ or prevent an increase in Aβ₄₂.

In one embodiment of the invention, it is contemplated that administration of the acetylcholine esterase inhibitor galantamine and one or more second compounds, e.g., (R)-2-(2-fluoro-4-biphenylyl)propionic acid can act in vivo, synergistically to treat and/or prevent Alzheimer's disease, dementia, MCI by lowering the amount of Aβ₄₂ that is present or would be present in the absence of such treatment. Amyloid β polypeptides are derived from amyloid precursor proteins (APPs). A variety of amyloid β polypeptides are known including Aβ₃₄, Aβ₃₇, Aβ₃₈, Aβ₃₉, and Aβ₄₀. Increased Aβ₄₂ levels are associated with Alzheimer's disease, dementia, MCI. Thus, by lowering the amounts of Aβ₄₂, a treatment is provided for combating Alzheimer's disease and/or MCI. It is contemplated that the combination of (R)-2-(2-fluoro-4-biphenylyl)propionic acid and galantamine can synergistically lessen the progression of symptoms of AD (or the rate of increase in the symptoms).

According to another embodiment, the invention provides a method of lowering Aβ₄₂ levels to a greater extent than inhibiting COX-1, COX-2, or a combination thereof In particular, the method of this embodiment comprises administering, to a patient in need of treatment, an effective amount of the acetylcholine esterase inhibitor galantamine (or a pharmaceutically acceptable salt, ester, or prodrug thereof) and the one or more second compounds(or a pharmaceutically acceptable salt, ester, or prodrug thereof), e.g., (R)-2-(2-fluoro-4-biphenylyl)propionic acid, wherein the effective amount of composition is capable of lowering Aβ₄₂, while not substantially affecting or inhibiting the activity of at least one isoform of COX. Thus, the method of this embodiment involves the lowering of Aβ₄₂ levels while not substantially inhibiting the activity of COX-1, COX-2, or both COX-1 and COX-2. The method of this embodiment can be used to treat and/or prevent Alzheimer's disease, MCI, dementia, and/or other neurodegenerative disorders. In one aspect of this embodiment, the effective amount of the one or more second compounds, e.g., (R)-2-(2-fluoro-4-biphenylyl)propionic acid and the acetylcholine esterase inhibitor galantamine reduces Aβ₄₂ levels or production of Aβ₄₂ by at least 1, 2, 5, 10, 15, 20, 25, 30, 40, or 50 or more percent while inhibiting COX-1, COX-2, or both COX-1 and COX-2 by less than 1, 2, 5, 10, 15, 20, 25, 30, 40, 50, 60, 70, 80, or 90 percent. In another aspect of this embodiment, the effective amount of the second compound, e.g., (R)-2-(2-fluoro-4-biphenylyl)propionic acid and the acetylcholine esterase inhibitor galantamine lowers Aβ₄₂ by at least 5 percent while not substantially inhibiting COX-1 , COX-2, or both COX-1 and COX-2 activity or levels. In another preferred aspect of this embodiment, the effective amount of the R-NSAID, e.g., (R)-2-(2-fluoro-4-biphenylyl)propionic acid, and the acetylcholine esterase inhibitor galantamine, that is administered to an individual is such that it lowers Aβ₄₂ levels, and does not inhibit COX activity to a significant extent, e.g., the amount administered is below the in vivo IC₅₀ value for COX-1, COX-2 or both COX-1 and COX-2 and above the in vivo IC₅₀ value for Aβ₄₂ lowering activity. As used in this context, IC₅₀ refers to the concentration of compound or composition sufficient to inhibit COX activity by 50% (COX-1, COX-2, or both COX-1 and COX-2) or reduce Aβ₄₂ levels (or rates of production) by 50%. An “effective amount” according to one aspect of this embodiment, can also be viewed in terms of ED₅₀ parameters, binding constants, dissociation constants, and other pharmacological parameters, e.g., the amount administered is below the ED₅₀ value for COX-1, COX-2 or both COX-1 and COX-2 and above the ED₅₀ value for Aβ₄₂. It is noted that the effective amount of the compound does not necessarily have to be above an IC₅₀ or ED₅₀ for Aβ₄₂ lowering and below the IC₅₀ or ED₅₀ for COX inhibition. That is, the “effective amount” can be at some intermediate value such that Aβ₄₂ levels (or rates of production) are lowered to a greater extent than inhibition of COX-1, COX-2 or both COX-1 and COX-2. In one aspect, the method of this embodiment is thought to avoid the liability of adverse side effects associated with COX-1 and COX-2 inhibitors.

In another embodiment, the combination therapy of the invention provides a lessening in decline in cognitive function is at least 25% as compared to individuals treated with placebo, more preferably at least 40%, and even more desirably at least 60%. For example, an individual treated with placebo having probable mild-to-moderate Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time.

In another embodiment, the invention provides a method of lowering Aβ₄₂ levels and increasing Aβ₃₈ levels, while not affecting Aβ₄₀ levels. The method of this embodiment comprises administering, to an individual in need of such treatment, an effective amount of the acetylcholine esterase inhibitor galantamine (or a pharmaceutically acceptable salt, ester, or prodrug thereof) and one or more second compounds (or a pharmaceutically acceptable salt, ester, or prodrugs thereof), e.g., (R)-2-(2-fluoro-4-biphenylyl)propionic acid. The method according to this embodiment is useful for preventing and treating Alzheimer's disease. It is also contemplated that the method of this embodiment is useful for treating and preventing other disorders such as MCI, dementia, other neurodegenerative disorders. The Aβ₄₂ lowering method of this embodiment can also increase the levels of other Aβ proteins smaller than Aβ₄₀, including Aβ₃₄, Aβ₃₇, Aβ₃₈, and Aβ₃₉.

In another embodiment, the invention relates to a method of preventing Alzheimer's disease. According to this embodiment, a method for preventing Alzheimer's disease is provided which comprises administering, to an individual in need of such treatment, a prophylactically effective amount the acetylcholine esterase inhibitor galantamine (or a pharmaceutically acceptable salt, ester, or prodrug thereof) and one or more second compounds (or a pharmaceutically acceptable salt, ester, or prodrug thereof), e.g., (R)-2-(2-fluoro-4-biphenylyl)propionic acid. The method of this embodiment is useful for preventing the symptoms of Alzheimer's disease, the onset of Alzheimer's disease, and/or the progression of the disease.

The invention provides, in yet another embodiment, a method of decreasing cognitive decline in a patient in need of such treatment. The method of this embodiment involves treating an individual desiring (or needing) a slowing or decrease in decline in cognitive function, with an effective amount of the acetylcholine esterase inhibitor galantamine (or a pharmaceutically acceptable salt, ester, or prodrug thereof) and one or more second compounds (or a pharmaceutically acceptable salt, ester, or prodrug thereof), i.e., (R)-2-(2-fluoro-4-biphenylyl)propionic acid.

In one embodiment, a patient suspected of having mild-to-moderate Alzheimer's disease is identified using diagnostic techniques readily available to the skilled practitioner (e.g., MMSE score of >15 and <26, has a diagnosis of dementia according to DSM IV (TR) and/or meets the NINCDS-ADRDA criteria for probable AD). The patient is then administered, on a daily basis, or twice daily basis (or any other acceptable dosing regime, e.g., thrice daily dosing), an Alzheimer's disease treating therapeutically effective amount of (R)-2-(2-fluoro-4-biphenylyl)propionic acid and the acetylcholine esterase inhibitor galantamine. The daily dosage of (R)-2-(2-fluoro-4-biphenylyl)propionic acid is from about 5 mg to about 4000 mg, from about 50 mg to about 3500 mg, from about 200 to about 3000 mg. The daily dosage of the acetylcholine esterase inhibitor is as follows (or the molar equivalent of the active ingredient if in the form of another salt form): from about 1 mg to about 40 mg of galantamine hydrobromide, from about 2 mg to about 36 mg of galantamine hydrobromide, from about 2 mg to about 30 mg of galantamine hydrobromide, from about 2 mg to about 20 mg of galantamine hydrobromide or from about 2 mg to about 16 mg of galantamine hydrobromide; from about 2 mg to about 10 mg of galantamine hydrobromide. In one specific aspect of this embodiment, 400 mg or more of (R)-2-(2-fluoro-4-biphenylyl)propionic acid is administered per day to the individual. In one specific aspect of this embodiment, 600 mg or more of (R)-2-(2-fluoro-4-biphenylyl)propionic acid is administered per day to the individual. In one specific aspect of this embodiment, 800 mg or more of (R)-2-(2-fluoro-4-biphenylyl)propionic acid is administered per day to the individual. In one specific aspect of this embodiment, 1000 mg or more of (R)-2-(2-fluoro-4-biphenylyl)propionic acid is administered per day to the individual. In one specific aspect of this embodiment, 1200 mg or more of (R)-2-(2-fluoro-4-biphenylyl)propionic acid. In one specific aspect of this embodiment, 1600 mg or more of (R)-2-(2-fluoro-4-biphenylyl)propionic acid is administered per day to the individual. In one specific aspect of this embodiment, 1600 mg of (R)-2-(2-fluoro-4-biphenylyl)propionic acid is administered per day to the individual. In one specific aspect of this embodiment, 36 mg of galantamine hydrobromide is administered per day to the individual. In one specific aspect of this embodiment, 24 mg of galantamine hydrobromide is administered per day to the individual. Unless indicated elsewhere, these recommended doses can be used for the other embodiments of the invention. Individuals having mild-to-moderate Alzheimer's disease, mild Alzheimer's disease, MCI, and prodromal Alzheimer's disease according can be treated with the above-recommended daily doses for 24 weeks or more, 36 weeks or more, 48 weeks or more, or 52 weeks or more, with the combination of (R)-2-(2-fluoro-4-biphenylyl)propionic acid and the acetylcholine esterase inhibitor galantamine. Alternatively, the patient can be started on the acetylcholine esterase inhibitor and titrated to the appropriate dose, and then treated with R-NSAID (i.e., (R)-2-(2-fluoro-4-biphenylyl)propionic acid) in combination with the acetylcholine esterase inhibitor. Desirably, the combination can be formulated in a single dosage form such as a tablet, capsule, caplet, or liquid for oral administration. The individual components of the combination ((R)-2-(2-fluoro-4-biphenylyl)propionic acid and acetylcholine esterase inhibitor) can also be administered separately, i.e., a tablet of (R)-2-(2-fluoro-4-biphenylyl)propionic acid and a tablet having the acetylcholine esterase inhibitor galantamine.

In one specific embodiment, the individual in need of treatment is administered 800 mg (R)-2-(2-fluoro-4-biphenylyl)propionic acid and 5 mg galantamine hydrobromide twice daily. In another embodiment, the individual in need of treatment is administered 800 mg (R)-2-(2-fluoro-4-biphenylyl)propionic acid and 4 mg galantamine hydrobromide twice daily. In yet another embodiment, the individual in need of treatment is administered 800 mg (R)-2-(2-fluoro-4-biphenylyl)propionic acid and 3 mg galantamine hydrobromide twice daily. In one embodiment, the individual in need of treatment is administered 800 mg (R)-2-(2-fluoro-4-biphenylyl)propionic acid and 2.5 mg galantamine hydrobromide twice daily. In another embodiment, the individual in need of treatment is administered 800 mg (R)-2-(2-fluoro-4-biphenylyl)propionic acid and 2 mg galantamine hydrobromide twice daily. In still another embodiment, the individual in need of treatment is administered 800 mg (R)-2-(2-fluoro-4-biphenylyl)propionic acid and 1 mg galantamine hydrobromide twice daily.

In some embodiments the amount of a particular ingredient (e.g., active pharmaceutical ingredient (API)) includes molar equivalents of the active ingredients if formulated as a different salt form (or alternatively, a bio-equivalent amount of the pharmaceutically acceptable salt).

In another aspect of the invention, a method for treating Alzheimer's disease is provided which involves administering to a patient an Aβ₄₂ lowering effective amount of a compound (i.e., (R)-2-(2-fluoro-4-biphenylyl)propionic acid) and galantamine.

In addition to using (R)-2-(2-fluoro-4-biphenylyl)propionic acid and acetylcholine esterase inhibitor, the invention includes using pharmaceutically acceptable prodrugs, pharmaceutically active metabolites, pharmaceutically acceptable esters, pharmaceutically acceptable derivatives, and pharmaceutically acceptable salts of such compounds.

Prodrugs and active metabolites of compound may be identified using routine techniques known in the art. See, e.g., Bertolini, G et al., J. Med. Chem., 40, 2011-2016 (1997); Shan, D. et al., J. Pharm. Sci., 86 (7), 756-767; Bagshawe K., Drug Dev. Res., 34, 220-230 (1995); Bodor N; Advance in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

While not wishing to be bound by theory, it is believed that the combination of (R)-2-(2-fluoro-4-biphenylyl)propionic acid and acetylcholine esterase inhibitor is capable of slowing the rate of death of neurons. Accordingly, it is also believed that the combination of R-NSAID and the acetylcholine esterase inhibitor acts in vivo to treat and/or prevent Alzheimer's disease and MCI by slowing the rate of death of neurons that is present or would be present in the absence of such treatment.

Patient Population

Any individual having, or suspected of having, a neurodegenerative disorder, such as Alzheimer's disease, can be treated using the compositions and methods of the present invention. Individuals who would particularly benefit from the compositions and methods of the invention include those individuals diagnosed as having mild to moderate Alzheimer's disease according to a medically-accepted diagnosis, such as, for example the NINCDS-ADRDA criteria. Progression of the disease may be followed by medically accepted measure of cognitive function, such as, for example, the Mini-Mental State Exam (MMSE; see Mohs et al. Int. Psychogeriatr. 8:195-203 (1996)); ADAS-Cog (Alzheimer Disease Assessment Scale-Cognitive; see Galasko et al. Alzheimer Dis Assoc Disord, 11 suppl 2:S33-9 (1997)); Behavioral Pathology in Alzheimer's Disease Rating Scale (BEHAVE-AD); Blessed Test; CANTAB—Cambridge Neuropsychological Test Automated Battery; CERAD (The Consortium to Establish a Registry for Alzheimer's Disease) Clinical and Neuropsychological Tests (includes MMSE); Clock Draw Test; Cornell Scale for Depression in Dementia (CSDD); Geriatric Depression Scale (GDS); Neuropsychiatric Inventory (NPI); the 7 Minute Screen; the Alzheimer's Disease Cooperative Study Activities of Daily Living scale (ADCS-ADL; see McKhann et al. Neurology 34:939-944 (1984)); the DSM-IV (Diagnostic and Statistical Manual of Mental Disorders—Fourth Edition (DSM-IV), published by the American Psychiatric Association, Washington D.C., 1994); or the NINCDS-ADRDA criteria (see Folstein et al. J. Psychiatr. Res. 12:189-198 (1975)). Individuals diagnosed as having probable AD can be identified as having a mild-to-moderate form of the disease by an accepted measure of cognitive function such as the MMSE. In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993). The severity of AD is generally determined by one of the initial tests provided above. For example, MMSE scores of 26-19 indicate mild AD, while scores from 18-10 indicate moderate AD.

Diagnoses of Alzheimer's disease based on these tests are recorded as presumptive or probable, and may optionally be supported by one or more additional criteria. For example, a diagnosis of Alzheimer's disease may be supported by evidence of a family history of AD; non-specific changes in EEG, such as increased slow-wave activity; evidence of cerebral atrophy on CT with progression documented by serial observation; associated symptoms such as depression, insomnia, incontinence, delusions, illusions, hallucinations, catastrophic verbal, emotional or physical outbursts, sexual disorders, weight loss, and/or attendant neurologic abnormalities, such as increased muscle tone, myoclonus or gait disorder, etc.

Additionally, amyloid deposits, generally associated with AD, may be detected through the use of positron emission tomography (PET) using an amyloid-specific tracer such as Pittsburgh Compound-B (PIB). See Klunk et al., Ann. Neurol. 55(3):306-309 (2004). Increased amyloid deposits in the frontal, parietal, temporal and occipital cortices, and in the striatum, relative to normal brain tissue, as visualized, for example by PIB, support a diagnosis of AD. Generally, a greater number and density of amyloid deposits indicates more advanced AD.

Additionally, the invention, is some embodiments, relates to identifying an individual who is experiencing a decrease in the ratio of Aβ42/Aβ40 ratio in cerebral spinal fluids (CSF) levels and treating said individual with a combination of the acetylcholine esterase inhibitor galantamine and the one or more second compounds, as described elsewhere in this application. Method of monitoring CSF levels of Aβ42 and Aβ40 are known to the skilled artisan and described herein.

The invention encompasses the treatment of an individual having mild to moderate AD, to the extent that individual has AD, whether or not one or more non-AD neurodegenerative diseases or conditions are previously, concurrently or subsequently diagnosed.

The compounds and methods of the present invention are useful for individuals who have received prior medication for AD, as well as individuals who have received no prior medication for AD, and is useful for individuals currently receiving medication for AD other than (R)-2-(2-fluoro-4-biphenylyl)propionic acid, and for individuals not receiving medication for AD other than (R)-2-(2-fluoro-4-biphenylyl)propionic acid.

Individuals of any age may be treated by the methods of the invention, with the pharmaceutical compositions of the invention; however, the invention encompasses specific embodiments for treating or preventing Alzheimer's disease in individuals between the ages of 45 and 100. In other various specific embodiments, individuals treated by the therapeutic or prophylactic methods of the invention may be from 55 to 70 years of age, 60 to 80 years of age, 55 to 65 years of age, 60 to 75 years of age, 65 to 80 years of age, 55 to 60 years of age, 60 to 65 years of age, 65 to 70 years of age, 70 to 75 years of age, 75 to 80 years of age, or 80 years old and older.

Thus, in one embodiment, the invention provides a method of treating an individual known or suspected of having Alzheimer's disease comprising administering an effective amount of (R)-2-(2-fluoro-4-biphenylyl)propionic acid and galantamine. In a specific embodiment, said individual is diagnosed as having mild to moderate Alzheimer's disease. In another specific embodiment, the individual is diagnosed by a cognitive test as having mild-to-moderate AD. In yet another embodiment, said cognitive test is the Mini-Mental State Exam (MMSE). In another specific embodiment, said individual has a score in said MMSE of from 26 to 19, inclusive. In another more specific embodiment, said individual has a score in said MMSE of from 18 to 10, inclusive. In another specific embodiment, said individual has a score in said MMSE of from 26 to 10, inclusive. In another specific embodiment, said individual has a score in said MMSE of from 18 or more, 19 or more, 20 or more, 21 or more, 22 or more, 23 or more, 24 or more, or 25 or more.

In another embodiment, said individual is concurrently taking a non-drug substance for the treatment of Alzheimer's disease. In a specific embodiment, said non-drug substance is an anti-oxidant. In another embodiment, said anti-oxidant is vitamin C or vitamin E. In yet another embodiment, said vitamin C is taken in a dose of 500-1000 mg per dose of (R)-2-(2-fluoro-4-biphenylyl)propionic acid. In another embodiment, said vitamin E is taken in a dose of 400-800 IU per dose of (R)-2-(2-fluoro-4-biphenylyl)propionic acid. In this regard, the invention encompasses the use of one or more such anti-oxidants as an adjunct to therapy for Alzheimer's disease, and not primarily as a nutritional supplement.

In another embodiment, the invention provides a method of treating an individual diagnosed as having mild-to-moderate Alzheimer's disease comprising administering an effective amount of (R)-2-(2-fluoro-4-biphenylyl)propionic acid, wherein said individual has, prior to taking (R)-2-(2-fluoro-4-biphenylyl)propionic acid, is taking another drug (i.e., galantamine) for the treatment of Alzheimer's disease. In another embodiment, said individual has, prior to taking (R)-2-(2-fluoro-4-biphenylyl)propionic acid and galantamine, has taken a non-drug substance for the treatment of Alzheimer's disease. In a specific embodiment, said non-drug substance is an anti-oxidant. In another specific embodiment, said anti-oxidant is vitamin C or vitamin E. In yet another specific embodiment, said vitamin C is taken in a dose of 500-1000 mg per dose. In yet another specific embodiment, said vitamin E is taken in a dose of 400-800 IU per dose. In this regard, the invention encompasses the use of one or more such anti-oxidants as an adjunct to therapy for Alzheimer's disease, and not primarily as a nutritional supplement.

Although any individual having, or suspected of having, Alzheimer's disease may be treated with (R)-2-(2-fluoro-4-biphenylyl)propionic acid and galantamine as described elsewhere herein, certain patient subpopulations may be identified that would especially benefit from the use of (R)-2-(2-fluoro-4-biphenylyl)propionic acid and galantamine. For example, the invention encompasses a preferred method wherein (R)-2-(2-fluoro-4-biphenylyl)propionic acid and galantamine is used in individuals who do not have: (1) a history in the past 2 years of epilepsy, focal brain lesion, head injury with loss of consciousness and/or immediate confusion after the injuries; (2) DSM-IV (TR) criteria for any major psychiatric disorder including psychosis, major depression, bipolar disorder, alcohol or substance abuse; (3) a history of hypersensitivity to flurbiprofen or other NSAIDs including COX-2 specific inhibitors; (4) a history of upper GI bleeding requiring transfusion or surgery within the past 3 years; (5) active gastric or duodenal ulcer disease; (6) a history of NSAID-associated ulcers; (7) active malignancy, or a history of active malignancy, except for basal cell carcinoma or squamous cell carcinoma of the skin; (8) chronic or acute renal, hepatic or metabolic disorder defined by creatinine>1.5 mg/dL, AST>2.5×Upper Limit of Normal (ULN); or ALT>2.5×ULN; uncontrolled cardiac conditions (New York Heart Association Class III or IV); (9) current anticoagulant therapy such as warfarin; or (10) current treatment with any CYP2C9 inhibitor (for example, amiodarone, fluconazole, fluvoxamine, isoniazid, phenylbutazone, probenicid, sulfamethoxazole, sulfaphenazole, trimethoprim, zafirlukast; danshen (Salvia miltiorrhiza); Lycium barbarum) or the CYP2C9 substrates fluvastatin, tolbutamide, or glyburide (glibenclamide); or who do not show chronic use of NSAIDs at any dose or aspirin>325 mg per day.

In yet another embodiment, the invention provides a method of slowing cognitive decline in an individual suspected of having mild cognitive impairment (MCI) comprising administering to the individual an effective amount of (R)-2-(2-fluoro-4-biphenylyl)propionic acid and galantamine. Mild cognitive impairment is a clinical condition between normal aging and Alzheimer's disease characterized by memory loss greater than expected for the particular age of the individual yet the individual does not meet the currently accepted definition for probable Alzheimer's disease. See, e.g., Petersen et al Arch. Neurol. 58:1985-1992 (2001); Petersen Nature Rev. 2:646-653 (2003); and Morris et al. J Mol. Neuro. 17:101-118 (2001). Thus, according to one aspect of the invention, an individual suspected of having or diagnosed with MCI is treated twice daily with a composition having from 400 mg to about 1200 mg of (R)-2-(2-fluoro-4-biphenylyl)propionic acid per dose in combination with a therapeutically effective amount of galantamine for at least 4 weeks, at least 4 months, preferably at least 8 months, and more desirably at least 1 year. Typically, patients having MCI first complain of or have a loss of memory. Preferably, an individual associated with the patient can corroborate the memory deficit. Furthermore, general cognition is not sufficiently impaired to cause concern about more widespread cognitive disorder and although daily living activities may be affected that are not significantly impaired and the patients are not demented. Individuals having or suspected of having MCI that are treated according to this embodiment can expect to slow cognitive decline and/or progression to probable AD, mild AD, and or mild-to-moderate AD.

The decline in cognitive function can be characterized by cognition tests. It is preferred that the lessening in decline in cognitive function is at least 25% as compared to individuals treated with placebo, at least 40%, or at least 60%. For example, an individual treated with placebo having probably mild-to-moderate Alzheimer's disease is expected to score approximately 5.5 points higher on the ADAS-cog test after a specified period of time (e.g., 1 year) whereas an individual treated with a composition of the invention for the same period of time will score only approximately 3.3 points higher on the ADAS-cog scale, i.e., will show 60% of the decline in cognitive function relative to untreated individuals, or 2.2 points higher i.e., will show 40% of the decline in cognitive function relative to untreated individuals, when treated for the same specified period of time.

Definitions

As used herein, the term “acetylcholine esterase inhibitors” refers to a class of pharmaceuticals known to inhibit the activity of the enzyme acetylcholine esterase, thereby increasing brain levels of acetylcholine. The skilled artisan recognizes that the acetylcholine esterase inhibitors include active ingredient and is not limited to one particular salt form. Galantamine is chemically know as (4aS, 6R,8aS)4a,5,9,10,11,-12-hexahydro-3-methoxy-11-methyl-6H-benzofuro [3,3a,2ef][2]benzapin-6-ol and is formulated as the hydrobromide. Galantamine hydrobromide has an empirical formula of C₁₇H₂₁NO₃.HBr.

As used herein, the term “preventing an increase in a symptom” refers to both not allowing a symptom to increase or worsen, as well as reducing the rate of increase in the symptom. For example, a symptom can be measured as the amount of particular disease marker, i.e., a protein. Preventing an increase, according to the definition provided herein, means that the amount of the protein does not increase or that the rate at which it increases is reduced.

As used herein, the term “treating Alzheimer's disease” refers to a slowing of or a reversal of the progress of the disease in an individual that has been diagnosed as having, or has one or more indicia of, Alzheimer's disease, as diagnosed by a test of cognition. Treating Alzheimer's disease includes reducing, lessening or improving one or more of the symptoms of the disease.

As used herein, the term “preventing Alzheimer's disease” refers to a slowing of, or stopping, the onset of the disease or of one or more of the symptoms thereof. In particular, the term means slowing or stopping the onset of one or more aspects of Alzheimer's disease that would otherwise lead to a diagnosis of at least mild Alzheimer's disease on one or more tests of cognition.

As used herein, the term “(R)-2-(2-fluoro-4-biphenylyl)propionic acid” refers to the R-enantiomer of the non-steroidal anti-inflammatory drug flurbiprofen. Desirably, the formulations of the invention are substantially free of (S)-2-(2-fluoro-4-biphenylyl)propionic acid. In one aspect, at least 90% by weight (R)-2-(2-fluoro-4-biphenylyl)propionic acid to 10% by weight or less of (S)-2-(2-fluoro-4-biphenylyl)propionic acid of the total 2-(2-fluoro-4-biphenylyl)propionic acid (S+R) is in the pharmaceutical composition. In another aspect, at least 95% by weight (R)-2-(2-fluoro-4-biphenylyl)propionic acid to 5% by weight or less of (S)-2-(2-fluoro-4-biphenylyl)propionic acid of the total 2-(2-fluoro-4-biphenylyl)propionic acid (S+R) is in the pharmaceutical composition. In yet another aspect, at least 99% by weight (R)-2-(2-fluoro-4-biphenylyl)propionic acid to 1% by weight or less of (S)-2-(2-fluoro-4-biphenyl)propionic acid of the total 2-(2-fluoro-4-biphenylyl)propionic acid (S+R) is in the pharmaceutical composition. In yet another aspect, at least 99.9% by weight (R)-2-(2-fluoro-4-biphenylyl)propionic acid to 0.1% by weight or less of (S)-2-(2-fluoro-4-biphenylyl)propionic acid of the total 2-(2-fluoro-4-biphenylyl)propionic acid (S+R) is in the pharmaceutical composition. In one aspect, the (R)-2-(2-fluoro-4-biphenylyl)propionic acid is tarenflurbil.

As used herein, the term “unit dosage form” refers to a physically discrete unit, such as a capsule or tablet suitable as a unitary dosage for a human patient.

As used herein, the term “dose” or “dosage” refers the amount of active ingredient that an individual takes or is administered at one time. For example, an 800 mg (R)-2-(2-fluoro-4-biphenylyl)propionic acid dose refers to, in the case of a twice-daily dosage regimen, a situation where the individual takes 800 mg (R)-2-(2-fluoro-4-biphenylyl)propionic acid in the morning and 800 mg (R)-2-(2-fluoro-4-biphenylyl)propionic acid in the evening. The 800 mg (R)-2-(2-fluoro-4-biphenylyl)propionic acid dose can be divided into two or more dosage units, e.g., two 400 mg (R)-2-(2-fluoro-4-biphenylyl)propionic acid tablets or two 400 mg (R)-2-(2-fluoro-4-biphenylyl)propionic acid capsules. The examples describe in this definition are not intended to be limiting and are merely to illustrate various specific doses or dosages.

As used herein, “decline,” when used to characterize a disease such as Alzheimer's, or a symptom or marker thereof, means a worsening or progression of the disease, symptom or marker thereof over time from less-advanced to more-advanced. In the case of Alzheimer's disease, a decline indicates a worsening or increase in the severity of one or more behavioral, cognitive, biochemical or clinical parameters of the condition. “Decline” also indicates a progression of one or more scores on a cognition test that indicate a worsening of the condition, regardless of whether the actual, raw scores increase or not.

As used herein, “Alzheimer's disease” and “AD” are equivalent. “A pharmaceutically acceptable prodrug” is a compound that may be converted under physiological conditions or by solvolysis to the specified compound or to a pharmaceutically acceptable salt of such compound.

“A pharmaceutically active metabolite” is intended to mean a pharmacologically active product produced through metabolism in the body of a specified compound or salt thereof. Metabolites of a compound may be identified using routine techniques known in the art and their activities determined using tests such as those described herein.

“A pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

Additional Combination Therapy

The invention further provides additional combination therapy strategies for treating neurodegenerative disorders such as Alzheimer's disease, MCI, and dementia. According to this aspect of the invention, an individual in need of treatment is administered an effective amount of (1) galantamine, (2) one or more second compounds (e.g., (R)-2-(2-fluoro-4-biphenylyl)propionic acid), and (3) one or more compounds selected from the group consisting of NSAIDs, COX-2 inhibitors (cyclooxygenase-2), β-secretase inhibitors, γ-secretase inhibitors, NMDA antagonists (i.e., memantine), and GABA-A alpha inverse agonist (see WO 00/27382, WO 96/25948, WO 98/50385 which are herein incorporated by reference in there entireties). NMDA receptor antagonists for combination therapy are memantine, adamantane, amantadine, an adamantane derivative, dextromethorphan, dextrorphan, dizocilpine, ibogaine, ketamine, and remacemide. The combination therapy of the invention is thought to provide a synergistic effect in reducing Aβ₄₂ levels and is surprisingly thought to be especially effective for treating and preventing neurodegenerative disorders including Alzheimer's disease, dementia, and MCI. The invention further encompasses compositions comprising the combination of active ingredients of this aspect of the invention.

According to another aspect of the invention, an individual in need of such treatment is administered an effective amount of (R)-2-(2-fluoro-4-biphenylyl)propionic acid, galantamine, and at least one NSAID. According to a preferred aspect of this embodiment the NSAID is selected from the group consisting of 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, indomethacin, naproxen, etolodac, tiaprofenic, suprofen, ketorolac, pirprofen, indoprofen, benoxaprofen, oxaprozin, diflunisal, and nabumetone.

The treatment regime used in the combination therapy can involve administration of a composition comprising the combination of active ingredients, the concomitant administration of separate compositions, each comprising at least one active ingredient. Furthermore, the administration of the active ingredients can be performed at different times and/or different routes. For example, a composition having one active ingredient can be administered in the morning, and a composition having the other active ingredients can be administered in the evening. Another example would involve the administration of a composition having two active ingredients orally while the third active ingredient is administered intravenously.

Preparation of the Compounds of the Invention

The compounds of the invention can be prepared by a variety of art known procedures. In one aspect, the one or more second compounds employed in the compositions and methods disclosed herein can be chosen from (R)-2-(2-fluoro-4-biphenylyl)propionic acid, (R)-2-(4-isobutyl-phenyl)-propionic acid, (R)-2-(3-benzoylphenyl)-propionic acid, (R)-5-benzoyl-2,3-dihydro-1H-pyrrolizine-1-carboxylic acid, (R)-5-benzoyl-alpha-methyl-2-thiopheneacetic acid, (R)-2-[4-(2-thienylcarbonyl)phenyl]propanoic acid, (R)-6-chloro-alpha-methylcarbazole-2-acetic acid, (R)-2-[3-Chloro-4-(2,5-dihydro-pyrrol-1-yl)-phenyl]-propionic acid, (R)-4-(1,3-dihydro-1-oxo-2H-isoindol-2-yl)-alpha-methylbenzeneacetic acid, and (R)-2-[2-(4-chlorophenyl)benzoxazol-5-yl]propionic acid. The one or more second compounds can also be a cyclized derivative of an arylpropionic acid, such as (R)-5-benzoyl-2,3-dihydro-1H-pyrrolizine-1-carboxylic acid, or an arylacetic acid, such as (R)-1,8-diethyl-1,3,4,9-tetrahydropyrano(3,4-b)indole-1-acetic acid. Descriptions of specific these compounds and their preparation can be found in various publications. (R)-2-(4-isobutyl-phenyl)-propionic acid is described in U.S. Pat No. 6,255,347. 2-(3-benzoylphenyl)-propionic acid is described in U.S. Pat. No. 3,641,127. 2-(2-fluoro-4-biphenylyl)propionic acid is described in U.S. Pat. No. 3,755,427. 5-benzoyl-2,3-dihydro-1H-pyrrolizine-1-carboxylic acid is described in U.S. Pat. No. 4,089,969.

A large number of the compounds (i.e., the one or more second compounds) useful according to the invention are commercially available either in the form of racemic mixtures or as optically pure enantiomers. For example, the following racemates can be obtained through Sigma Chemical Co.: 2-(3-benzoylphenyl)-propionic acid, 2-(2-fluoro-4-biphenylyl)propionic acid, and 1,8-diethyl-1,3,4,9-tetrahydropyrano(3,4-b)indole-1-acetic acid, as well as others. Additionally, many commercial sources exist for the stereospecific R-isomers. (R)-2-(3-benzoylphenyl)-propionic acid, (R)-2-(2-fluoro-4-biphenylyl)propionic acid and (R)-5-benzoyl-2,3-dihydro-1H-pyrrolizine-1-carboxylic acid, for example, are available through Sepracor, Inc.; (R)-5-benzoyl-2,3-dihydro-1H-pyrrolizine-1-carboxylic acid can be obtained as the sodium salt through Sigma Chemical Co.; (R)-1,8-diethyl-1,3,4,9-tetrahydropyrano(3,4-b)indole-1-acetic acid is available from Wyeth-Ayerst; (R)-5-benzoyl-alpha-methyl-2-thiopheneacetic acid is available through Roussel (France, Canada, Switzerland, Spain, Denmark, Italy); (R)-2-[4-(2-thienylcarbonyl)phenyl]propanoic acid is manufactured by McNiel Pharmaceuticals; (R)-6-chloro-alpha-methylcarbazole-2-acetic acid is available from Roche; (R)-2-[3-chloro-4-(2,5-dihydro-pyrrol-1-yl)-phenyl]-propionic acid is available through Ciba (France, Belgium, Denmark); (R)-4-(1,3-dihydro-1-oxo-2H-isoindol-2-yl)-alpha-methylbenzeneacetic acid can be obtained through Carlo Elba (Italy, U.K.); and (R)-2-[2-(4-chlorophenyl)benzoxazol-5-yl]propionic acid is manufactured by Eli Lilly Co.

Preparation of (R)-2-(2-fluoro-4-biphenylyl)propionic acid and Galantamine

(R)-2-(2-fluoro-4-biphenylyl)propionic acid compositions are disclosed in, e.g., U.S. Pat. No. 5,200,198 to Geisslinger et al.

Methods of resolving (R)-2-(2-fluoro-4-biphenylyl)propionic acid from the racemate are disclosed in U.S. Pat. No. 5,599,969 to Hardy et al. which discloses contacting the racemates with α-methylbenzylamine salt in a solvent mixture of toluene and methanol, followed by recrystallization of the diastereomer salt. The diastereomer salts are then separated to give the resolved flurbiprofen enantiomers. U.S. Pat. No. 4,209,638 to Boots Co. discloses a process for resolving 2-arylproprionic acids which include flurbiprofen by mixing the racemate with a chiral organic nitrogenous base under certain conditions followed by recovery and separation of the diastereomeric salts. Other patents disclosing processes for resolving racemic arylproprionic acids include U.S. Pat. No. 4,983,765 to PAZ; U.S. Pat. No. 5,015,764 to Ethyl Corp.; U.S. Pat. No. 5,235,100 to Ethyl Corp.; U.S. Pat. No. 5,574,183 to Albemarle Corp.; U.S. Pat. No. 5,510,519 to Sumitomo Chemical Company.

Methods of tableting (R)-2-(2-fluoro-4-biphenylyl)propionic acid and arylproprionic acids are disclosed in, e.g., U.S. Pat. No. 5,667,807 to Hurner et al.; U.S. Pat. No. 5,565,613 to Geisslinger at al.; U.S. Pat. No. 6,471,991 to Robinson et al.; and U.S. Pat. No. 6,379,707 to Vladyka et al.

The acetylcholine esterase inhibitors galantamine (Reminyl®) is from Ortho-McNeil Neurologics, Inc., Titusville, N.J. Galantamine is disclosed in U.S. Pat. Nos. 4,663,318, and 6,099,863 both of which are hereby incorporated by reference in their entireties. All of the patents referenced in this section are hereby incorporated by reference in their entireties.

Aβ42 Lowering Agents

The Aβ42 lowering agents for use in the invention can be a known Aβ42 lowering agents such as (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. Examples of Aβ42 lowering agents for use in the combination formulations and treatments of the invention are given in, e.g., WO 01/78721, WO 2004/073705, WO 2004/064771, and WO 2004/074232 (each of which is herein incorporated by reference).

Aβ42 lowering agents include, but are not limited to, those having the following Formulae: 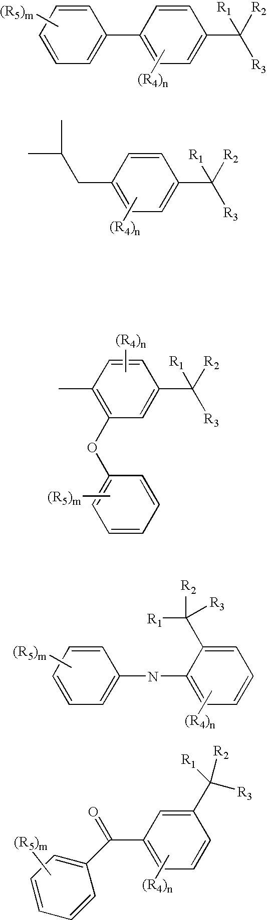
Where R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);
R₂ is chosen from —CH₃, —CH₂CH₃, —CH₂CH₂CH₃, and —CH₂CH₂CH₂CH₃, (or can be taken together with R₂ to give a cyclopropyl ring, a cyclobutyl ring, a cyclopentyl ring, or a cyclohexyl ring);
R₃ is chosen from —COOH, —COOR₆, —CONH₂, —CONHR₆, —CONR₆R₇, —CONHSO₂R₆, tetrazolyl, and a —COOH bioisostere;
R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₆ is chosen from —CH₃, —CH₂CH₃, —CH₂CH₂CH₃, and —CH₂CH₂CH₂CH₃.
R₇ is chosen from —CH₃, —CH₂CH₃, —CH₂CH₂CH₃, and —CH₂CH₂CH₂CH₃.
M is an integer chosen from 0, 1, 2, and 3.
N is an integer chosen from 0, 1, 2, and 3.

Examples of compounds (i.e., the one or more second compounds) for use in the invention include those as shown above (and those listed below), including enantiomers, diastereomers, racemates, and pharmaceutically acceptable salts thereof. The compounds described in this invention disclosure can be made by an ordinary artisan skilled in the art of organic chemistry synthesis.

Additional Aβ42 lowering agents for use in the invention include, but are not limited to, 2-methyl-2 (2-fluoro-4′-trifluoromethylbiphen-4-yl)propionic acid; 2-methyl-2 (2-fluoro-4′cyclohexyl biphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro-4′-hydroxybiphenyl-4-yl)cyclopropanecarboxylic acid; 1-[2-fluoro-4′-(tetrahydropyran-4-yloxy)biphenyl-4-yl]-cyclopropane-carboxylic acid;1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid;1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl)benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-ter phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1′-tert-phenyl]-4-yl)-cyclopropane-carboxylic acid; 1-(2,2′-difluoro-4″-hydroxy[1,1′:4′,1′-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis (trifluoromethyl)biphen-4-yl)propionic acid; 2-(4′-cyclohexyl-2-fluorobiphen-4-yl)propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

Aβ₄₂ lowering agents can be identified by a number of methods. To identify Aβ₄₂ lowering agents that reduce APP processing, a biological composition having an APP processing activity (i.e. an activity that processes APP into various Aβ forms, one of which is Aβ₄₂), is incubated with APP under conditions in which APP processing occurs. To identify Aβ₄₂ lowering agents that increase Aβ₄₂ catabolism, a biological composition having Aβ₄₂ catabolic activity is incubated with Aβ₄₂ under conditions in which Aβ₄₂ catabolism occurs. Depending on the nature of the biological composition, the APP or Aβ₄₂ substrate can be added to the biological composition, or, each or both can be a component of the biological composition. APP processing or Aβ₄₂ catabolism is allowed to take place in the presence or absence of the candidate Aβ₄₂ lowering agent. The level of Aβ₄₂ generated from APP processing or the level of Aβ₄₂ remaining after the catabolic reaction, in the presence and absence of the candidate Aβ₄₂ lowering agent, is determined and compared. Aβ₄₂ lowering agents useful for treating AD are those that reduce the level of Aβ₄₂ either by reducing APP processing into Aβ₄₂ or by enhancing Aβ₄₂ catabolism and increasing Aβ₃₈ production. The biological composition having an APP processing and/or catabolic activity can be a cell-free biological sample. For example, a cell-free biological sample can be a purified or partially purified enzyme preparation; it also can be a cell lysate generated from cells able to process APP into Aβ₄₂ or from cells able to catabolize Aβ₄₂. Cell lysates can be prepared using known methods such as, for example, sonication or detergent-based lysis. In the case of an enzyme preparation or cell lysate, APP can be added to the biological composition having the APP processing activity, or Aβ₄₂ can be added to the biological composition having Aβ₄₂ catabolic activity.

In addition, the biological composition can be any mammalian cell that has an APP processing activity as well as a nucleic acid vector encoding APP. Alternatively, the biological composition can be any mammalian cell that has Aβ catabolic activity as well as a nucleic acid vector or a viral nucleic acid-based vector containing a gene that encodes Aβ₄₂. The vector typically is an autonomously replicating molecule, a molecule that does not replicate but is transiently transfected into the mammalian cell, or a vector that is integrated into the genome of the cell. Typically, the mammalian cell is any cell that can be used for heterologous expression of the vector-encoded APP or Aβ₄₂ in tissue culture. For example, the mammalian cell can be a Chinese hamster ovary (CHO) cell, a fibroblast cell, or a human neuroglioma cell. The mammalian cell also can be one that naturally produces APP and processes it into Aβ₄₂, or one that naturally produces and catabolizes Aβ₄₂.

Further, the biological composition can be an animal such as a transgenic mouse that is engineered to over-express a form of APP that then is processed into Aβ₄₂. Alternatively, the animal can be a transgenic mouse that is engineered to over-express Aβ₄₂. Animals can be, for example, rodents such as mice, rats, hamsters, and gerbils. Animals also can be rabbits, dogs, cats, pigs, and non-human primates, for example, monkeys.

To perform an in vitro cell-free assay, a cell-free biological sample having an activity that can process APP into Aβ₄₂ is incubated with the substrate APP under conditions in which APP is processed into various Aβ forms including Aβ₄₂ (see Mclendon et al. (2000) FASEB 14:2383-2386). Alternatively, a cell-free biological sample having an activity that can catabolize Aβ₄₂ is incubated with the substrate Aβ₄₂ under conditions in which Aβ₄₂ is catabolized. To determine whether a candidate Aβ₄₂ lowering agent has an effect on the processing of APP into Aβ₄₂ or the catabolism of Aβ₄₂, two reactions are compared. In one reaction, the candidate Aβ₄₂ lowering agent is included in the processing or catabolic reaction, while in a second reaction, the candidate Aβ₄₂ lowering agent is not included in the processing or catabolic reaction. Levels of the different Aβ forms produced in the reaction containing the candidate Aβ₄₂ lowering agent are compared with levels of the different Aβ forms produced in the reaction that does not contain the candidate Aβ₄₂ lowering agent.

The different Aβ forms can be detected using any standard antibody based assays such as, for example, immunoprecipitation, western hybridization, and sandwich enzyme-linked immunosorbent assays (ELISA). Different Aβ forms also can be detected by mass spectrometry; see, for example, Wang et al. (1996) J Biol Chem 271:31894-902. Levels of Aβ species can be quantified using known methods. For example, internal standards can be used as well as calibration curves generated by performing the assay with known amounts of standards.

In vitro cell-based assays can be used determine whether a candidate Aβ₄₂ lowering agent has an effect on the processing of APP into Aβ₄₂ or an effect on catabolism of Aβ₄₂. Typically, cell cultures are treated with a candidate Aβ₄₂ lowering agent. Then the level of Aβ₄₂ in cultures treated with a candidate Aβ₄₂ lowering agent is compared with the level of Aβ₄₂ in untreated cultures. For example, mammalian cells expressing APP are incubated under conditions that allow for APP expression and processing as well as Aβ₄₂ secretion into the cell supernatant. The level of Aβ₄₂ in this culture is compared with the level of Aβ₄₂ in a similarly incubated culture that has been treated with the candidate Aβ₄₂ lowering agent. Alternatively, mammalian cells expressing Aβ₄₂ are incubated under conditions that allow for Aβ₄₂ catabolism. The level of Aβ₄₂ in this culture is compared with the level of Aβ₄₂ in a similar culture that has been treated with the candidate Aβ₄₂ lowering agent.

In vivo animal studies also can be used to identify Aβ₄₂ lowering agents useful for treating AD. Typically, animals are treated with a candidate Aβ₄₂ lowering agent and the levels of Aβ₄₂ in plasma, CSF, and/or brain are compared between treated animals and those untreated. The candidate Aβ₄₂ lowering agent can be administered to animals in various ways. For example, the candidate Aβ₄₂ lowering agent can be dissolved in a suitable vehicle and administered directly using a medicine dropper or by injection. The candidate Aβ₄₂ lowering agent also can be administered as a component of drinking water or feed. Levels of Aβ in plasma, cerebral spinal fluid (CSF), and brain are determined using known methods. For example, levels of Aβ₄₂ can be determined using sandwich ELISA or mass spectrometry in combination with internal standards or a calibration curve. Plasma and CSF can be obtained from an animal using standard methods. For example, plasma can be obtained from blood by centrifugation, CSF can be isolated using standard methods, and brain tissue can be obtained from sacrificed animals.

When present in an in vitro or in vivo APP processing or Aβ₄₂ catabolic reaction, Aβ₄₂ lowering agents reduce the level of Aβ₄₂ generated by APP processing or remaining following Aβ catabolism. For example, in an in vitro cell-free assay, the level of Aβ₄₂ is reduced due to either a reduction of APP processing or an increase in Aβ₄₂ catabolism in the presence the Aβ₄₂ lowering agent. In an in vitro cell culture study, a reduction in the level of Aβ₄₂ secreted into the supernatant results from the effect of the Aβ₄₂ lowering agent on either a reduction in processing of APP into Aβ₄₂ or an increased catabolism of Aβ₄₂. Similarly, in animal studies, a reduction in the level of Aβ₄₂ that can be detected in plasma, CSF, or brain is attributed to the effect of the Aβ₄₂ lowering agent on either a reduction in the processing of APP into Aβ₄₂ or an increase in the catabolism of Aβ₄₂. The level of Aβ₄₂ can be reduced by a detectable amount. For example, treatment with an Aβ₄₂ lowering agent leads to a 0.5, 1, 3, 5, 7, 15, 20, 40, 50, or more than 50% reduction in the level of Aβ₄₂ generated by APP processing or remaining following Aβ₄₂ catabolism when compared with that in the absence of the Aβ₄₂ lowering agent. Preferably, treatment with the Aβ₄₂ lowering agent leads to at least a 20% reduction in the level of Aβ₄₂ generated when compared to that in the absence of Aβ₄₂ lowering agent. More preferably, treatment with an Aβ₄₂ lowering agent leads to at least a 40% reduction the level of Aβ₄₂ when compared to that in the absence of an Aβ₄₂ lowering agent.

Dosages, Formulations and Route of Administration

The active compounds of this invention are typically administered in combination with a pharmaceutically acceptable carrier through any appropriate routes such as parenteral, oral, or topical administration, in a therapeutically (or prophylactically) effective amount according to the methods set forth above. A preferred route of administration for use in the invention is oral administration.

Generally, the toxicity profile and therapeutic efficacy of the therapeutic agents can be determined by standard pharmaceutical procedures in suitable cell models or animal models. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but remains significantly below the LD₅₀ dosage level, as determined from cell or animal models.

Typically, the compounds and compositions for use in the invention can be effective at an amount of from about 0.05 mg to about 4000 mg per day, preferably from about 0. 1 mg to about 2000 mg per day. However, the amount can vary with the body weight of the patient treated and the state of disease conditions. The active ingredient may be administered at once, or may be divided into a number of smaller doses to be administered at predetermined intervals of time.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in the art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound for use in the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

The formulations and unit dosage forms of the invention can have a number of different ingredients. Depending on the dosage strength, a unit dosage form has an amount of active pharmaceutical ingredient(s) (API) sufficient for achieving a therapeutic effect in a target population. Additionally “inactive pharmaceutical ingredients” need to be present to achieve a therapeutically effect release of the API. Thus the amount and type of inactive ingredients help achieve a therapeutically effective release of the therapeutic agent. In one aspect of the invention, a tablet unit dosage form is provided having the following inactive ingredients: one or more disintegrants in an amount sufficient to facilitate break-up (disintegration) of the tablet after administration (e.g., provide an immediate release dissolution profile), one or more binders in an amount sufficient to impart adequate cohesiveness to the tablet and/or provide adequate free flowing qualities by formulation of granules of desired size/hardness, one or more diluents in an amount sufficient to impart satisfactory compression characteristics, one or more lubricants in an amount sufficient to provide an adequate flow rate of the granulation and/or prevent adhesion of the material to the die/punch, reduce interparticle friction, and/or facilitate ejection from the die, and if desired, optional ingredients.

The disintegration rate, and often the dissolution rate of a compacted solid pharmaceutical formulation in an aqueous environment (e.g., the patient's stomach) may be increased by the addition of a disintegrant to the formulation. Disintegrants include alginic acid, carboxymethylcellulose calcium, carboxymethylcellulose sodium (e.g., Ac-Di-Sol® Primellose®.), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Solid pharmaceutical formulations that are compacted into a dosage form, such as a tablet, may include excipients whose functions include helping to bind the active pharmaceutical ingredient and other excipients together after compression. Binders for solid pharmaceutical formulations include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methylcellulose (e.g. Methocel®), lactose, liquid glucose, magnesium aluminum silicate, maltodextrin, methylcellulose, polymethacrylates, povidone (e.g. Kollidon®, Plasdone®), pregelatinized starch, sodium alginate and starch. Glidants can be added to improve the flowability of a non-compacted solid formulation and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered formulation, the formulation is subjected to pressure from a punch and dye. Some excipients and active pharmaceutical ingredients have a tendency to adhere to the surfaces of the punch and dye, which can cause the product to have pitting and other surface irregularities. A lubricant can be added to the formulation to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Examples of diluents include, but are not limited to, calcium carbonate, calcium phosphate, calcium sulfate, cellulose, cellulose acetate, compressible sugar, confectioner's sugar, dextrates, dextrin, dextrose, ethyl cellulose, fructose, fumaric acid, glyceryl palmitostearate, hydrogenated vegetable oil, kaolin, lactitol, lactose, magnesium carbonate, magnesium oxide, maltodextrin, maltose, mannitol, medium chaim glyceride, microcrystalline cellulose, polydextrose, polymethylacrylates, simethicone, sodium alginate, sodium chloride, sorbitol, starch, pregelantized starch, sterilizable maize, sucrose, sugar spheres, talc, tragacanth, trehalose, and xylitol.

Examples of disintegrants include, but are not limited to, alginic acid, calcium phosphate, carboxymethyl cellulose calcium, carboxymethyl cellulose sodium, powdered cellulose, chitosan, crospovidone, docusate sodium, guar gum, hydroxylpropyl cellulose, magnesium aluminum silicate, methylcellulose, poidone, sodium alginate, sodium starch glycolate, starch, and pregelantinized starch.

Example of binders (binding agents) include, but are not limited to, acacia, alginic acid, carbomers, carboxymethyl cellulose sodium, carrageenan, cellulose acetate phthalate, ceratonia, chitosan, confectioners sugar, cottonseed oil, dextrates, dextrin, dextrose, ethylcellulose, gelatin, glucose, glyceryl behenate, guar gum, hydrogenated vegetable oil, hydroxyethyl cellulose, hydroxyethylmethyl cellulose, hydroxylpropyl cellulose, hypromellose, magnesium aluminum silicate, maltodextrin, maltodextrin, maltose, methylcellulose, microcrystalline cellulose, poloxamer, polydextrose, polyethylene oxide, polymethyl acrylates, povidone, sodium alginate, starch, pregelantized starch, stearic acid, sucrose, sunflower oil, and zein.

Examples of lubricants include, but are not limited to, calcium stearate, glycerin monostearate, glyceryl behenate, glyceryl palmitostearate, hydrogenated castor oil, hydrogenated vegetable oil, light mineral oil, magnesium lauryl sulfate, magnesium stearate, medium chain triglycerides, mineral oil, poloxamer, polyethylene glycol, sodium benzoate, sodium chloride, sodium lauryl sulfate, sodium stearyl fumarate, stearic acid, talc, and zinc stearate.

Examples of glidants include, but are not limited to, calcium phosphate, calcium silicate, cellulose powdered, colloidal silicon dioxide, magnesium silicate, magnesium trisilicate, silicon dioxide, starch, and talc.

Optional ingredients in the formulations of the invention include, but are not limited to, flavors, coloring agents, and stabilizers.

Flavoring agents and flavor enhancers make the dosage form more palatable to the patient. Common flavoring agents and flavor enhancers for pharmaceutical products that may be included in the formulation of the present invention include maltol, vanillin, ethyl vanillin, menthol, citric acid, fumaric acid, ethyl maltol and tartaric acid. Solid and liquid formulations may also be dyed using any pharmaceutically acceptable colorant to improve their appearance and/or facilitate patient identification of the product and unit dosage level.

In one embodiment, the tablet unit dosage form has a hardness of about 5 kp (kilopond) or more, about 7 kp or more, about 9 kp or more, about 11 kp or more, and about 13 kp or more to avoid excessive friability, and a hardness of about 20 kp or less, about 19 kp or less, about 18 kp or less, about 17 kp or less, and about 16 kp or less, is desirable to avoid subsequent difficulty in hydrating the tablet when exposed to gastric fluid. In some aspects of this embodiment, the hardness of the tablet unit dosage form is from 9 kp to 18 kp, 11 kp to 17 kp, and 13 kp to 17 kp. When hardness is in an acceptable range, tablet friability is typically less than about 1.0%, preferably less than about 0.8% and more preferably less than about 0.5%, in a standard test. Some issues that may cause variations in tablet hardness are inconsistent tablet weight, particle size variations, poor powder compressibility, and insufficient binder level.

The tablet unit dosage forms of the invention have a friability of less than about 1%, less than about 0.9%, less than about 0.8%, less than about 0.7%, less than about 0.6%, less than about 0.5%, and less than about 0.4% (all at 100 rev).

### EXAMPLES

### Example 1

### Co-Formulation of (R)-2-(2-fluoro-4-biphenylyl)propionic acid with an acetylcholine esterase Inhibitor

(R)-2-(2-fluoro-4-biphenylyl)propionic acid Galantamine Tablets

| Ingredient | Amount |
|---|---|
| (R)-2-(2-fluoro-4-biphenylyl)propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 16 mg |

(R)-2-(2-fluoro-4-biphenylyl)propionic acid Galantamine Tablets

| Ingredient | Amount |
|---|---|
| (R)-2-(2-fluoro-4-biphenylyl)propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 10 mg |

(R)-2-(2-fluoro-4-biphenylyl)propionic acid Galantamine Tablets

| Ingredient | Amount |
|---|---|
| (R)-2-(2-fluoro-4-biphenylyl)propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 8 mg |

(R)-2-(2-fluoro-4-biphenylyl)propionic acid Galantamine Tablets

| Ingredient | Amount |
|---|---|
| (R)-2-(2-fluoro-4-biphenylyl)propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 5 mg |

The tablets are prepared using art known procedures and the amounts ingredients listed above can be modified (e.g., coated) to obtain an improved formulation.

### Example 2

### Co-Formulation of 2-(4-isobutyl-phenyl)-2-methyl Propionic acid with an acetylcholine esterase Inhibitor

2-(4-isobutyl-phenyl)-2-methyl propionic acid Galantamine Tablets

| Ingredient | Amount |
|---|---|
| 2-(4-isobutyl-phenyl)-2-methyl propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 16 mg |

2-(4-isobutyl-phenyl)-2-methyl propionic acid Galantamine Tablets

| Ingredient | Amount |
|---|---|
| (R)-2-(2-fluoro-4-biphenylyl)propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 10 mg |

2-(4-isobutyl-phenyl)-2-methyl propionic acid Galantamine Tablets

| Ingredient | Amount |
|---|---|
| 2-(4-isobutyl-phenyl)-2-methyl propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 8 mg |

2-(4-isobutyl-phenyl)-2-methyl propionic acid acid
Galantamine Tablets

| Ingredient | Amount |
|---|---|
| 2-(4-isobutyl-phenyl)-2-methyl propionic | 400 mg |
| acid | |
| Microcrystalline Cellulose | 392 mg |
| Colloidal Silicon Dioxide | 4 mg |
| Magnesium Stearate | 4 mg |
| Galantamine hydrobromide | 5 mg |

### Example 3

### Co-Formulation of 2-(2-fluoro-1,1′-biphenyl-4-yl)-2-methylpropionic acid with an acetylcholine esterase Inhibitor

2-(2-fluoro-1,1′-biphenyl-4-yl)-2-methylpropionic acid
Galantamine Tablets

| Ingredient | Amount |
|---|---|
| 2-(2-fluoro-1,1′-biphenyl-4-yl)-2- | 400 mg |
| methylpropionic acid | |
| Microcrystalline Cellulose | 392 mg |
| Colloidal Silicon Dioxide | 4 mg |
| Magnesium Stearate | 4 mg |
| Galantamine hydrobromide | 16 mg |

2-(2-fluoro-1,1′-biphenyl-4-yl)-2-methylpropionic acid
Galantamine Tablets

| Ingredient | Amount |
|---|---|
| 2-(2-fluoro-1,1′-biphenyl-4-yl)-2- | 400 mg |
| methylpropionic acid | |
| Microcrystalline Cellulose | 392 mg |
| Colloidal Silicon Dioxide | 4 mg |
| Magnesium Stearate | 4 mg |
| Galantamine hydrobromide | 10 mg |

2-(2-fluoro-1,1′-biphenyl-4-yl)-2-methylpropionic
acid Galantamine Tablets

| Ingredient | Amount |
|---|---|
| 2-(2-fluoro-1,1′-biphenyl-4-yl)-2- | 400 mg |
| methylpropionic acid | |
| Microcrystalline Cellulose | 392 mg |
| Colloidal Silicon Dioxide | 4 mg |
| Magnesium Stearate | 4 mg |
| Galantamine hydrobromide | 8 mg |

2-(2-fluoro-1,1′-biphenyl-4-yl)-2-methylpropionic
acid Galantamine Tablets

| Ingredient | Amount |
|---|---|
| 2-(2-fluoro-1,1′-biphenyl-4-yl)-2- | 400 mg |
| methylpropionic acid | |
| Microcrystalline Cellulose | 392 mg |
| Colloidal Silicon Dioxide | 4 mg |
| Magnesium Stearate | 4 mg |
| Galantamine hydrobromide | 5 mg |

### Example 4

### Treatment of Alzheimer's Disease with (R)-2-(2-fluoro-4-biphenylyl)propionic acid and Galantamine

The (R)-2-(2-fluoro-4-biphenylyl)propionic acid can be administered twice daily as tablets containing 800 mg of active ingredient or as a capsule containing 800 mg of the active ingredient. A higher dose can be administered to the patient in need of such treatment which can involve the patient taking, e.g., a 1000 mg dose of (R)-2-(2-fluoro-4-biphenylyl)propionic acid in the morning and a 1000 mg dose of (R)-2-(2-fluoro-4-biphenylyl)propionic acid in the evening. Galantamine (as the hydrochloride) can be administered twice daily as tablets containing 16 mg of galantamine hydrobromide (or 12 mg twice daily). Typically, for the treatment of mild-to-moderate Alzheimer's disease, an individual is diagnosed by a doctor as having the disease using a suitable combination of observations. One criterion indicating a likelihood of mild-to-moderate Alzheimer's disease is a score of about 15 to about 26 on the MMSE test (in a specific sub-group the patient has an MMSE of from 20-26, inclusive). Another criteria indicating mild-to-moderate Alzheimer's disease is a decline in cognitive function. (R)-2-(2-fluoro-4-biphenylyl)propionic acid can also be administered in liquid or dosage forms. The dosages can also be divided or modified, and taken with or without food. For example, the 800 mg dose can be divided into two 400 mg tablets or capsules (or four 200 mg unit dosage forms).

Depending on the stage of the disease, (R)-2-(2-fluoro-4-biphenylyl)propionic acid can also be administered twice daily in liquid, capsule, or tablet dosage forms where the dose has various amounts of (R)-2-(2-fluoro-4-biphenylyl)propionic acid (i.e., 850 mg, 750 mg, 700 mg, 650 mg, 600 mg, 550 mg, 500 mg, 450 mg, 350 mg, 300 mg, 250 mg, 200 mg, 150 mg, and 100 mg). Again, the dosages can also be divided or modified, and taken with or without food.

Alternatively, galantamine and (R)-2-(2-fluoro-4-biphenylyl)propionic acid can be co-formulated into a single dosage form, i.e., liquid, tablet, capsule, etc.

Patients having mild-to-moderate Alzheimer's disease undergoing the treatment regimen of this example with (R)-2-(2-fluoro-4-biphenylyl)propionic acid doses of about 800 mg (BID) and galantamine 16 mg (BID; or 12 mg BID) can experience a lessening in decline of cognitive function (as measured by the ADAS-cog or CDR sum of boxes), plaque pathology, and/or biochemical disease marker progression.

### Example 5

### Detection of Amyloid Beta with Biosource Elisa Kit (Camarillo, Calif.)

The present invention provides combination compositions and methods for lowering Aβ₄₂ levels. To test whether the combinations are capable of modulating Aβ levels, a sandwich enzyme-linked immunosorbent assay (ELISA) is employed to measure secreted Aβ (Aβ₄₂ and/or Aβ₄₀) levels. In this example, H4 cells expressing wide type APP695 are seeded at 200,000 cells/per well in 6 well plates, and incubated at 37° C. with 5% CO₂ overnight. Cells are treated with 1.5 ml medium containing vehicle (DMSO) or a test compounds at 1.25 μM, 2.5 μM, 5.0 μM and 10.0 μM (as well as other concentration if desirable) concentration for 24 hours or 48 hours. The supernatant from treated cells is collected into eppendorf tubes and frozen at −80 ° C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and dilutions of samples to be analyzed is added. The plate is incubated for 2 hours while shaking on an orbital plate shaker at RT. The plate is then washed 4 times with Working Wash Buffer and patted dry on a paper towel. Detection Antibody Solution is poured into a reservoir and 100 μL/well of Detection Antibody Solution is immediately added to the plate. The plate is incubated at RT for 2 hours while shaking and then washed four times with Working Wash Buffer and patted dry on a paper towel. Secondary Antibody Solution is then poured into a reservoir and 100 μL/well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix resulting in a change of solution color from blue to yellow. The absorbance of each well is read at 450 nm having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

### Example 6

### Combination Treatment of Animals to Determine the Combination's Effect on Memory and Alzheimer's Disease Progression

The present invention provides combination compositions and methods for treating or preventing Alzheimer's disease. To test the effect of compositions of the present invention on memory and Alzheimer's disease, TG2576 mice that overexpress APP(695) with the “Swedish” mutation (APP695NL) are used. Mice overexpressing APP(695) with the “Swedish” mutation develop memory deficits and plaques with age, making them suitable for examining the effect of compounds ((R)-2-(2-fluoro-4-biphenylyl)propionic acid and galantamine) on memory and Alzheimer's Disease. The test compounds are administered daily for two weeks to test groups of the TG2576 mice in age groups of: 1) 4-5 months, 2) 6-11 months, 3) 12-18 months, and 4) 20-25 months. Groups of control TG2576 mice of corresponding ages are not administered the compound. Both control and test groups then have memory tested in a version of the Morris water maze (Morris, J. Neurosci. Methods, 11:47-60 (1984)) that is modified for mice. The water maze contains a metal circular pool of about 40 cm in height and 75 cm in diameter. The walls of the pool have fixed spatial orientation clues of distinct patterns or shelves containing objects. The pool is filled with room temperature water to a depth of 25 cm and an escape platform is hidden 0.5 cm below the surface of the 25-cm-deep water at a fixed position in the center of one of the southwest quadrant of pool. The test and control mice are trained for 10 days in daily sessions consisting of four trials in which the mouse starts in a different quadrant of the pool for each trial. The mice are timed and given 60 seconds to find the escape platform in the pool. If the mice have not found the escape platform after 60 seconds, they are guided into it. The mice are then allowed to rest on the platform for 30 seconds and the amount of time it takes the mice to find the platform is recorded. Probe trials are run at the end of the trials on the 4th, 7th, and 10th days of training, in which the platform is removed and the mice are allowed to search for the platform for 60 sec. The percentage of time spent in the quadrant where the platform was in previous trials is calculated.

In training trials, the time it takes test group mice to reach the escape platform is compared to the time taken by control group mice of corresponding ages. In probe trials, the percentage of time spent by test group mice in the quadrant where the platform was in previous trials is compared to the percentage time spent by control mice. Quicker location of the escape platform in training trials and/or an increased percentage time spent in the previous quadrant of the maze during probe trials is indicative of spatial learning and memory. Because memory loss is a hallmark of Alzheimer's disease, test mice that have better learning and memory when compared to control mice indicate that the combination can be effective in treating or slowing Alzheimer's disease and/or its symptoms.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A unit dosage form comprising a combination of (R)-2-(2-fluoro-4-biphenylyl)propionic acid or a pharmaceutically acceptable salt or ester thereof and an acetylcholine esterase inhibitor or a pharmaceutically acceptable salt or ester thereof.

2. The unit dosage form of claim 1 wherein said acetylcholine esterase inhibitor is galantamine.

3. The unit dosage form of claim 1 wherein (R)-2-(2-fluoro-4-biphenylyl)propionic acid or a pharmaceutically acceptable salt or ester thereof is present in an amount from 100 mg to 1000 mg.

4. The unit dosage form of claim 2 wherein galantamine or a pharmaceutically acceptable salt or ester thereof is present in an amount from 1 to 40 mg.

5. The unit dosage form of claim 1 wherein (R)-2-(2-fluoro-4-biphenylyl)propionic acid or a pharmaceutically acceptable salt or ester thereof is present in an amount from 200 mg to 800 mg.

6. The unit dosage form of claim 2 wherein galantamine or a pharmaceutically acceptable salt or ester thereof is present in an amount from 2 mg to 30 mg.

7. The unit dosage form of claim 1 wherein (R)-2-(2-fluoro-4-biphenylyl)propionic acid or a pharmaceutically acceptable salt or ester thereof is present in an amount from 300 mg to 500 mg.

8. The unit dosage form of claim 2 wherein galantamine or a pharmaceutically acceptable salt or ester thereof is present in an amount from 2 mg to 20 mg.

9. The unit dosage form according to claims 1, wherein said unit dosage form is chosen from a tablet, a capsule, and a caplet.

10. The unit dosage form of claim 1, further comprising microcrystalline cellulose.

11. A method of treating mild Alzheimer's disease in an individual comprising identifying an individual having mild Alzheimer's disease and administering to the individual an Alzheimer's disease treating effective amount of (R)-2-(2-fluoro-4-biphenylyl)propionic acid or a pharmaceutically acceptable salt or ester thereof and an acetylcholine esterase inhibitor or a pharmaceutically acceptable salt or ester thereof.

12. The method of claim 11 wherein the acetylcholine esterase inhibitor is a galantamine.

13. The method of 12 wherein galantamine and (R)-2-(2-fluoro-4-biphenylyl)propionic acid are co-formulated.

14. The method of claim 12 wherein galantamine and (R)-2-(2-fluoro-4-biphenylyl)propionic acid are co-administered.

15. The method of claim 12 wherein said individual is titrated to a stable dose of galantamine prior to treatment with (R)-2-(2-fluoro-4-biphenylyl)propionic acid.

16. The method of claim 12 wherein 1 mg to 40 mg of galantamine, or a pharmaceutically acceptable salt or ester, thereof is administered per day.

17. The method of claim 12 wherein 32 mg of galantamine, or a pharmaceutically acceptable salt or ester thereof, is administered per day.

18. The method of claim 12 wherein 24 mg of galantamine, or a pharmaceutically acceptable salt or ester thereof, is administered per day.

19. The method of claim 12 wherein 400 or more mg of (R)-2-(2-fluoro-4-biphenylyl)propionic acid, or a pharmaceutically acceptable salt or ester thereof, is administered per day.

20. The method of claim 12 wherein 600 or more mg of (R)-2-(2-fluoro-4-biphenylyl)propionic acid, or a pharmaceutically acceptable salt or ester thereof, is administered per day.

21. The method of claim 12 wherein 800 or more mg of (R)-2-(2-fluoro-4-biphenylyl)propionic acid, or a pharmaceutically acceptable salt or ester thereof, is administered per day.

22. The method of claim 12 wherein 1600 or more mg of (R)-2-(2-fluoro-4-biphenylyl)propionic acid, or a pharmaceutically acceptable salt or ester thereof, is administered per day.

23. A co-formulation comprising a first compound which is galantamine or a pharmaceutically acceptable salt or ester thereof and a second compound which is an Aβ42 lowering agent or a pharmaceutically acceptable salt or ester thereof.

24. The co-formulation of claim 23 wherein said Aβ42 lowering agent is chosen from 5[1-(2-Fluoro-biphenyl-4-yl)-1-methyl-ethyl]-2H-tetrazole, 2-(4-isobutyl-phenyl)-2-methyl propionic acid, 2-(2-fluoro-1,1′-biphenyl-4-yl)-2-methylpropionic acid, 2-methyl-2 (2-fluoro-4′-trifluoromethylbiphen-4-yl)propionic acid, 2-methyl-2 (2-fluoro-4′cyclohexyl biphen-4-yl)propionic acid, 1-(2-fluoro-4′-trifluoromethylbiphenyl-4-yl)cyclopropanecarboxylic acid, 1-(4′-cyclohexyl-2-fluorobiphenyl-4-yl)cyclopropanecarboxylic acid, 1-(4′-benzyloxy-2-fluorobiphenyl-4-yl)cyclopropanecarboxylic acid, 1-(2-fluoro-4′-isopropyloxybiphenyl-4-yl)cyclopropanecarboxylic acid, 1-(2-fluoro-3′-trifluoromethoxybiphenyl-4-yl)cyclopropanecarboxylic acid, 1-(2-fluoro-4′-trifluoromethoxybiphenyl-4-yl)cyclopropanecarboxylic acid, 1-(2-fluoro-3′-trifluoromethylbiphenyl-4-yl)cyclopropanecarboxylic acid, 1-(4′-cyclopentyl-2-fluorobiphenyl-4-yl)cyclopropanecarboxylic acid, 1-(4′-cycloheptyl-2-fluorobiphenyl-4-yl)cyclopropanecarboxylic acid, 1-(2′-cyclohexyl-2-fluorobiphenyl-4-yl)cyclopropanecarboxylic acid, 1-(2-fluoro-4′-hydroxybiphenyl-4-yl)cyclopropanecarboxylic acid, 1-[2-fluoro-4′-(tetrahydropyran-4-yloxy)biphenyl-4-yl]-cyclopropane-carboxylic acid, 1-(2,3′,4′-trifluorobiphenyl-4-yl)cyclopropanecarboxylic acid, 1-(3′,4′-dichloro-2-fluorobiphenyl-4-yl)cyclopropanecarboxylic acid, 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid, 1-(4-benzo[b]thiophen-3-yl-3-fluorophenyl)cyclopropanecarboxylic acid, 1-(2-fluoro-4′-prop-2-inyloxy-biphenyl-4-yl)-cyclopropanecarboxylic acid, 1-(4′-cyclohexyloxy-2-fluoro-biphenyl-4-yl)-cyclopropanecarboxylic acid, 1-[2-fluoro-4′-(tetrahydropyran-4-yl)-biphenyl-4-yl]-cyclopropanecarboxylic acid, 1-[2-fluoro-4′-(4-oxo-cyclohexyl)-biphenyl-4-yl]-cyclopropanecarboxylic acid, 2-(2″-fluoro-4-hydroxy-[1,1′:4′,1″]tert-phenyl-4″-yl)-cyclopropanecarboxylic acid, 1-[4′-(4,4-dimethylcyclohexyl)-2-fluoro[1,1′-biphenyl]-4-yl]-cyclopropane-carboxylic acid, 1-[2-fluoro-4′-[[4-(trifluoromethyl)benzoyl]amino][1,1′-biphenyl]-4-yl]-cyclopropanecarboxylic acid, 1-[2-fluoro-4′-[[4-(trifluoromethyl)cyclohexyl]oxy][1,1′-biphenyl]-4-yl]-cyclopropanecarboxylic acid, 1-[2-fluoro-4′-[(3,3,5,5-tetramethylcyclohexyl)oxy][1,1′-biphenyl]-4-yl]-cyclopropanecarboxylic acid, 1-[4′-[(4,4-dimethylcyclohexyl)oxy]-2-fluoro[1,1′-biphenyl]-4-yl]-cyclopropanecarboxylic acid, 1-(2,3′,4″-trifluoro[1,1′:4′,1″-tert-phenyl]-4-yl)-cyclopropanecarboxylic acid, 1-(2,2′,4″-trifluoro[1,1′:4′,1″-tert-phenyl]-4-yl)-cyclopropanecarboxylic acid, 1-(2,3′-difluoro-4″-hydroxy[1,1′:4′,1″-tert-phenyl]-4-yl)-cyclopropane-carboxylic acid, 1-(2,2′-difluoro-4″-hydroxy[1,1′:4′,1″-tert-phenyl]-4-yl)-cyclopropane-carboxylic acid, 2-(2-fluoro-3′,5′-bis(chloro)biphen-4-yl)propionic acid amide, 2-(2-fluoro-4′-trifluoromethylbiphen-4-yl)propionic acid, 2-(2-fluoro-3′-trifluoromethylbiphen-4-yl)propionic acid, 2-(2-fluoro-3′,5′-bis(trifluoromethyl)biphen-4-yl)propionic acid, 2-(4′-cyclohexyl-2-fluorobiphen-4-yl)propionic acid, 2-(2-Fluoro-1,1′-biphenyl-4-yl)-2-methylpropanoic acid, 2-Methyl-2-(3-phenoxy-phenyl)-propionic acid, 2-(4-Isobutyl-phenyl)-2-methyl-propionic acid; 2-(6-Chloro-9H-carbazol-2-yl)-2-methyl-propionic acid, 2-[1-(4-Chloro-benzoyl)-5-methoxy-2-methyl-1H-indol-3-yl]-2-methyl-propionic acid, and 5-[1-(2-Fluoro-biphenyl-4-yl)-1-methyl-ethyl]-2H-tetrazole, or a pharmaceutically acceptable salt or ester thereof.

25. The co-formulation of claim 23 wherein galantamine or a pharmaceutically acceptable salt or ester thereof is present in an amount from 1 mg to 40 mg.
